(19) United States
(12) Patent Application Publication
(10) Pub. No.: US 2021/0023134 A1
(43) Pub. Date: Jan. 28, 2021

(54) METHODS OF ADMINISTERING CHIMERIC ANTIGEN RECEPTOR IMMUNOTHERAPY IN COMBINATION WITH 4-1BB AGONIST

(71) Applicants: Pfizer Inc., New York, NY (US); Kite Pharma, Inc., Santa Monica, CA (US)

(72) Inventors: William Y. GO, Thousand Oaks, CA (US); Adrian WOOLFSON, New York, NY (US)

(73) Assignees: Pfizer Inc., New York, NY (US); Kite Pharma, Inc., Santa Monica, CA (US)

(21) Appl. No.: 16/962,294

(22) PCT Filed: Jan. 15, 2019
(86) PCT No.: PCT/US2019/013595

§ 371 Date: Jul. 15, 2020

Related U.S. Application Data
(60) Provisional application No. 62/617,562, filed on Jan. 15, 2018.

(51) Int. Cl. A61K 35/17; A61P 35/00; C07K 16/28; C12N 15/87; G01N 33/50

(52) U.S. Cl. CPC A61K 35/17; A61P 35/00; G01N 33/5008; C12N 15/87; C07K 16/2878

(57) ABSTRACT

The disclosure provides a method of treatment of a B-cell lymphoma or leukemia, including diffuse large B-cell lymphoma (DLBCL) comprising a CD19-directed chimeric antigen receptor (CAR) genetically modified T-cell immunotherapy in combination with a 4-IBB (CD137) agonist. Some aspects of the disclosure relate to methods of treatment and monitoring following infusion of T-cell therapy provided herein.

### TECHNICAL FIELD

The present disclosure relates generally to T-cell therapies and more specifically to combination therapies of CD19-directed genetically modified T-cell immunotherapies comprising a chimeric antigen receptor (CAR) and a 4-1BB (CD137) agonist.

### INCORPORATION BY REFERENCE OF SEQUENCE LISTING

The Sequence Listing in an ASCII text file, named as 36991_Sequence_Listing.txt of 9 KB, created on Jul. 14, 2020, and submitted to the United States Patent and Trademark Office via EFS-Web, is incorporated herein by reference.

### BACKGROUND

Human cancers are by their nature comprised of healthy cells that have undergone a genetic or epigenetic conversion to become abnormal cancer cells. In doing so, cancer cells begin to express proteins and other antigens that are distinct from those expressed by healthy cells. These aberrant tumor antigens can be used by the body's innate immune system to specifically target and kill cancer cells. However, cancer cells employ various mechanisms to prevent immune cells, such as T- and B-lymphocytes, from successfully targeting cancer cells.

Chimeric antigen receptors (CARs), which comprise binding domains capable of interacting with a particular tumor antigen, allow T-cells induced to express them to target and kill cancer cells expressing the particular tumor antigen that they recognize.

4-1BB (also referred to as CD137, TNFRSF9, etc.) is a transmembrane protein of the Tumor Necrosis Factor receptor superfamily (TNFRS). 4-1BB promotes enhanced cellular proliferation, survival, and cytokine production (Croft, 2009, Nat Rev Immunol 9:271-285).

### SUMMARY

As described in detail below, the present disclosure is based, in part, on the surprising discovery that the administration methods disclosed herein lead to improved anti-CD19 CAR T-cell immunotherapy.

Any aspect or embodiment described herein may be combined with any other aspect or embodiment as disclosed herein, unless the context indicates otherwise. While the present invention has been described in conjunction with the detailed description thereof, the foregoing description is intended to illustrate and not limit the scope of the present invention, which is defined by the scope of the appended claims. Other aspects, advantages, and modifications are within the scope of the following claims.

In one aspect, the disclosure provides a method of treating a B-cell lymphoma or leukemia in a patient in need thereof comprising administering a CD19-directed genetically modified T-cell immunotherapy and a 4-1BB (CD137) agonist.

In some embodiments, the CD19-directed genetically modified T-cell immunotherapy, is genetically modified to express a chimeric antigen receptor (CAR) said CAR comprising an anti-CD19 single chain variable fragment (scFv) linked to CD28 and CD3-zeta co-stimulatory domains.

In some embodiments, the CD19-directed genetically modified T-cell immunotherapy is an autologous immunotherapy.

In some embodiments, the CD19-directed genetically modified T-cell immunotherapy is an allogenic immunotherapy.

In some embodiments, the T-cells are genetically modified ex vivo. In some embodiments, the T-cells are genetically modified by viral transduction. In some embodiments, the T-cells are genetically modified by retroviral transduction. In some embodiments, the T-cells are genetically modified by lentiviral transduction.

In some embodiments, the CD19-directed genetically modified T-cell immunotherapy is axicabtagene ciloleucel.

In some embodiments, the 4-1BB (CD137) agonist is an antigen binding molecule or fragment thereof.

In some embodiments, the 4-1BB (CD137) agonist is an isolated antibody, or antigen-binding portion thereof, comprising three CDRs of a VH region amino acid sequence as set forth in SEQ ID NO:1 and three CDRS of a VL region amino acid sequence set forth in SEQ ID NO: 3.

In some embodiments, the 4-1BB (CD137) agonist is an isolated antibody, or antigen-binding portion thereof, comprising: (a) a H-CDR1 as set forth in SEQ ID NO:5; (b) a H-CDR2 as set forth in SEQ ID NO:6; (c) a H-CDR3 as set forth in SEQ ID NO:7; (d) a L-CDR1 as set forth in SEQ ID NO:8; (e) a L-CDR2 as set forth in SEQ ID NO:9; and (f) a L-CDR3 as set forth in SEQ ID NO:10.

In some embodiments, the 4-1BB (CD137) agonist is a fully human monoclonal antibody.

In some embodiments, the 4-1BB (CD137) agonist comprises a VH region amino acid sequence as set forth in SEQ ID NO:1 and a VL region amino acid sequence as set forth in SEQ ID NO: 3.

In some embodiments, the 4-1BB (CD137) agonist comprises a heavy chain amino acid sequence as set forth in SEQ ID NO:2 and a light chain amino acid sequence as set forth in SEQ ID NO:4, with the proviso that the C-terminal lysine residue of SEQ ID NO:2 is optionally absent.

In some embodiments, the 4-1BB (CD137) agonist is utomilumab.

In some embodiments, the B-cell lymphoma or leukemia is selected from the group consisting of Acute Lymphoblastic Leukemia (ALL), AIDS-related lymphoma, ALK-positive large B-cell lymphoma, Burkitt's lymphoma, Chronic lymphocytic leukemia, CLL), Classical Hodgkin lymphoma, Diffuse large B-cell lymphoma (DLBCL), Primary Mediastinal Large B-cell Lymphoma (PMBCL), Follicular lymphoma, Intravascular large B-cell lymphoma, Large B-cell lymphoma arising in HHV8-associated multicentric Castleman's disease, Lymphomatoid granulomatosis, Lymphoplasmacytic lymphoma, Mantle cell lymphoma (MCL), Marginal zone B-cell lymphoma (MZL), Mucosa-Associated Lymphatic Tissue lymphoma (MALT), Nodal marginal zone B-cell lymphoma (NMZL), Nodular lymphocyte predominant Hodgkin's lymphoma, Non-Hodgkin's lymphoma, Plasmablastic lymphoma, Primary central nervous system lymphoma, Primary effusion lymphoma, Splenic marginal zone lymphoma (SMZL), and Waldenström's macroglobulinemia, relapsed or refractory large B-cell lymphoma, diffuse large B-cell lymphoma (DLBCL) not otherwise specified, high grade B-cell lymphoma, and DLBCL arising from follicular lymphoma.

In some embodiments, the B-cell lymphoma is selected from the group consisting of relapsed or refractory large B-cell lymphoma, diffuse large B-cell lymphoma (DLBCL) not otherwise specified, primary mediastinal large B-cell lymphoma (PMBCL), high grade B-cell lymphoma, and DLBCL arising from follicular lymphoma. Various additional lymphoma types are described in the 2016 revision of the World Health Organization classification of lymphoid neoplasms found at Swerdlow et al., Blood 2016 127:2375-2390; doi: https://doi.org/10.1182/blood-2016-01-643569.

In some embodiments, the B-cell lymphoma is relapsed or refractory diffuse large B-cell lymphoma.

In some embodiments, the CD19-directed genetically modified T-cell immunotherapy and the 4-1BB (CD137) agonist are administered after two or more lines of systemic therapy in a patient. In some embodiments, the CD19-directed genetically modified T-cell immunotherapy and the 4-1BB (CD137) agonist are administered to treatment-naïve patients. In some embodiments, the CD19-directed genetically modified T-cell immunotherapy and the 4-1BB (CD137) agonist are administered to patients who have not received other systemic therapy prior to administration of the CD19-directed genetically modified T-cell immunotherapy and the 4-1BB (CD137) agonist.

In some embodiments, the CD19-directed genetically modified T-cell immunotherapy is administered to the patient by intravenous infusion at a dose between about 1×10⁶ and about 2×10⁶ CAR-positive viable T-cells per kg body weight up to a maximum dose of about 1×10⁸ CAR-positive viable T-cells.

In some embodiments, the CD19-directed genetically modified T-cell immunotherapy is administered only once.

In some embodiments, the CD19-directed genetically modified T-cell immunotherapy is administered more than once.

In some embodiments, the 4-1BB (CD137) agonist is administered by intravenous infusion.

In some embodiments, the 4-1BB (CD137) agonist is administered at a dose ranging from about 1 mg to about 200 mg.

In some embodiments, the 4-1BB (CD137) agonist is administered at a dose of about 1 mg, about 10 mg, about 100 mg or about 200 mg. In some embodiments, the 4-1BB (CD137) agonist is administered at a dose ranging from about 1-200 mg, about 1-150 mg, about 1-125 mg, about 1-100 mg, about 10-200 mg, about 10-150 mg, about 10-125 mg, about 10-100 mg, about 25-200 mg, about 25-150 mg, about 25-125 mg, about 25-100 mg, about 30-200 mg, about 30-150 mg, about 30-125 mg, about 30-100 mg, about 50-200 mg, about 50-150 mg, about 50-125 mg, 50-100 mg, or about 100-200 mg.

In some embodiments, the CD19-directed genetically modified T-cell immunotherapy and the 4-1BB (CD137) agonist are administered simultaneously.

In some embodiments, the CD19-directed genetically modified T-cell immunotherapy is administered prior to the 4-1BB (CD137)) agonist.

In some embodiments, the first dose of the 4-1BB (CD137) agonist is administered the day following the CD19-directed genetically modified T-cell immunotherapy infusion.

In some embodiments, the CD19-directed genetically modified T-cell immunotherapy is administered after the 4-1BB (CD137) agonist.

In some embodiments, the 4-1BB (CD137) agonist dosing continues until patients demonstrate complete remission, non-response/progressive disease. In some embodiments, the 4-1BB (CD137)) agonist is administered for about 1 year.

In some embodiments, the 4-1BB (CD137) agonist is administered about every 4 weeks. In some embodiments, the 4-1BB (CD137) agonist is administered monthly. In some embodiments, the 4-1BB (CD137) agonist is administered about every 28 days. In some embodiments, the 4-1BB (CD137) agonist is administered about every 30 days.

In some embodiments, the patient is administered a conditioning chemotherapy regimen prior to administration of the CD19-directed genetically modified T-cell immunotherapy and the 4-1BB (CD137) agonist.

In one aspect, the present invention provides a CD19-directed genetically modified T-cell immunotherapy and a 4-1BB (CD137) agonist for use in a method of treating a B-cell lymphoma or leukemia in a patient in need thereof.

In one aspect, the present invention provides a method of treating a B-cell lymphoma or leukemia in a patient in need thereof comprising: (a) administering to the patient a CD19-directed genetically modified T-cell immunotherapy; (b) administering to the patient a 4-1BB (CD137) agonist; and (c) monitoring the patient following administration for signs and symptoms of an adverse reaction.

In some embodiments, the CD19-directed genetically modified T-cell immunotherapy is an autologous immunotherapy.

In some embodiments, the CD19-directed genetically modified T-cell immunotherapy is an allogenic immunotherapy.

In some embodiments, the T-cells are genetically modified ex vivo.

In some embodiments, the T-cells are genetically modified by viral transduction.

In some embodiments, the T-cells are genetically modified by retroviral transduction.

In some embodiments, the T-cells are genetically modified by lentiviral transduction.

In some embodiments, the CD19-directed genetically modified T-cell immunotherapy, is genetically modified to express a chimeric antigen receptor (CAR) said CAR comprising an anti-CD19 single chain variable fragment (scFv) linked to CD28 and CD3-zeta co-stimulatory domains.

In some embodiments, the CD19-directed genetically modified T-cell immunotherapy is axicabtagene ciloleucel.

In some embodiments, the 4-1BB (CD137) agonist is an antigen binding molecule or fragment thereof.

In some embodiments, the 4-1BB (CD137) agonist is an isolated antibody, or antigen-binding portion thereof, comprising three CDRs of a VH region amino acid sequence as set forth in SEQ ID NO:1 and three CDRS of a VL region amino acid sequence set forth in SEQ ID NO: 3.

In some embodiments, the 4-1BB (CD137) agonist is an isolated antibody, or antigen-binding portion thereof comprising: (a) a H-CDR1 as set forth in SEQ ID NO:5; (b) a H-CDR2 as set forth in SEQ ID NO:6; (c) a H-CDR3 as set forth in SEQ ID NO:7; (d) a L-CDR1 as set forth in SEQ ID NO:8; (e) a L-CDR2 as set forth in SEQ ID NO:9; and (f) a L-CDR3 as set forth in SEQ ID NO:10.

In some embodiments, the 4-1BB (CD137) agonist is a fully human monoclonal antibody.

In some embodiments, the 4-1BB (CD137) agonist comprises a VH region amino acid sequence as set forth in SEQ ID NO:1 and a VL region amino acid sequence as set forth in SEQ ID NO: 3.

In some embodiments, the 4-1BB (CD137) agonist comprises a heavy chain amino acid sequence as set forth in SEQ ID NO:2 and a light chain amino acid sequence as set forth in SEQ ID NO:4, with the proviso that the C-terminal lysine residue of SEQ ID NO:2 is optionally absent.

In some embodiments, the 4-1BB (CD137) agonist is utomilumab.

In some embodiments, the B-cell lymphoma or leukemia is selected from the group consisting of Acute Lymphoblastic Leukemia (ALL), AIDS-related lymphoma, ALK-positive large B-cell lymphoma, Burkitt's lymphoma, Chronic lymphocytic leukemia, CLL), Classical Hodgkin lymphoma, Diffuse large B-cell lymphoma (DLBCL), Primary Mediastinal Large B-cell Lymphoma (PMBCL), Follicular lymphoma, Intravascular large B-cell lymphoma, Large B-cell lymphoma arising in HHV8-associated multicentric Castleman's disease, Lymphomatoid granulomatosis, Lymphoplasmacytic lymphoma, Mantle cell lymphoma (MCL), Marginal zone B-cell lymphoma (MZL), Mucosa-Associated Lymphatic Tissue lymphoma (MALT), Nodal marginal zone B-cell lymphoma (NMZL), Nodular lymphocyte predominant Hodgkin's lymphoma, Non-Hodgkin's lymphoma, Plasmablastic lymphoma, Primary central nervous system lymphoma, Primary effusion lymphoma, Splenic marginal zone lymphoma (SMZL), and Waldenström's macroglobulinemia, relapsed or refractory large B-cell lymphoma, diffuse large B-cell lymphoma (DLBCL) not otherwise specified, high grade B-cell lymphoma, and DLBCL arising from follicular lymphoma.

In some embodiments, the B-cell lymphoma is selected from the group consisting of relapsed or refractory large B-cell lymphoma, diffuse large B-cell lymphoma (DLBCL) not otherwise specified, primary mediastinal large B-cell lymphoma, high grade B-cell lymphoma, and DLBCL arising from follicular lymphoma.

In some embodiments, the B-cell lymphoma is refractory diffuse large B-cell lymphoma.

In some embodiments, the adverse reaction is selected from the group consisting of cytokine release syndrome (CRS), a neurologic toxicity, a hypersensitivity reaction, a serious infection, a cytopenia and hypogammaglobulinemia.

In one aspect, the present invention provides a CD19-directed genetically modified T-cell immunotherapy and a 4-1BB (CD137) agonist for use in a method of treating a B-cell lymphoma or leukemia in a patient in need thereof comprising: (a) administering to the patient a CD19-directed genetically modified T-cell immunotherapy; (b) administering to the patient a 4-1BB (CD137) agonist; and (c) monitoring the patient following administration for signs and symptoms of an adverse reaction.

In one aspect, the present invention provides for a method of treating refractory diffuse large B-cell lymphoma in a patient in need thereof comprising: (a) administering to the patient a CD19-directed genetically modified T-cell immunotherapy; (b) administering to the patient a 4-1BB (CD137) agonist; and (c) monitoring the patient following administration for changes in markers of phenotype and activation of patient peripheral blood mononuclear cells (PBMCs).

In one aspect, the present invention provides a method of treating refractory diffuse large B-cell lymphoma after two or more lines of systemic therapy in a patient in need thereof comprising: (a) administering to the patient a CD19-directed genetically modified T-cell immunotherapy; (b) administering to the patient a 4-1BB (CD137) agonist; and (c) monitoring the patient following administration for changes in markers of phenotype and activation of patient peripheral blood mononuclear cells (PBMCs).

In some embodiments, the CD19-directed genetically modified T-cell immunotherapy is an autologous immunotherapy.

In some embodiments, the CD19-directed genetically modified T-cell immunotherapy is an allogenic immunotherapy.

In some embodiments, the T-cells are genetically modified ex vivo.

In some embodiments, the T-cells are genetically modified by retroviral transduction.

In some embodiments, the CD19-directed genetically modified T-cell immunotherapy, is genetically modified to express a chimeric antigen receptor (CAR) said CAR comprising an anti-CD19 single chain variable fragment (scFv) linked to CD28 and CD3-zeta co-stimulatory domains.

In some embodiments, the CD19-directed genetically modified T-cell immunotherapy is axicabtagene ciloleucel.

In some embodiments, the 4-1BB (CD137) agonist is an antigen binding molecule or fragment thereof.

In some embodiments, the 4-1BB (CD137) agonist is an isolated antibody, or antigen-binding portion thereof, comprising three CDRs of a VH region amino acid sequence as set forth in SEQ ID NO:1 and three CDRS of a VL region amino acid sequence set forth in SEQ ID NO: 3.

In some embodiments, the 4-1BB (CD137) agonist is an isolated antibody, or antigen-binding portion thereof comprising: (a) a H-CDR1 as set forth in SEQ ID NO:5; (b) a H-CDR2 as set forth in SEQ ID NO:6; (c) a H-CDR3 as set forth in SEQ ID NO:7; (d) a L-CDR1 as set forth in SEQ ID NO:8; (e) a L-CDR2 as set forth in SEQ ID NO:9; and (f) a L-CDR3 as set forth in SEQ ID NO:10.

In some embodiments, the 4-1BB (CD137)) agonist is a fully human monoclonal antibody.

In some embodiments, the 4-1BB (CD137) agonist comprises a VH region amino acid sequence as set forth in SEQ ID NO:1 and a VL region amino acid sequence as set forth in SEQ ID NO: 3.

In some embodiments, the 4-1BB (CD137) agonist comprises a heavy chain amino acid sequence as set forth in SEQ ID NO:2 and a light chain amino acid sequence as set forth in SEQ ID NO:4, with the proviso that the C-terminal lysine residue of SEQ ID NO:2 is optionally absent.

In some embodiments, the 4-1BB (CD137) agonist is utomilumab.

In some embodiments, the markers of phenotype and activation of patient PBMCs comprise a pan T-cell marker, cytotoxic T-cell marker, differentiation T-cell marker, differentiation marker, IL-2 receptor, activation marker, PD1, 4-1BB, helper T-cell marker, granulocyte marker, B-cell marker, monocyte/macrophage marker, NK cell marker, and/or axicabtagene ciloleucel identification.

In some embodiments, the markers of phenotype and activation of patient PBMCs are monitored by a panel comprising antibodies to CD3, CD8, CD45RA, CCR7, CD122, CD27, CD28, CD95, and/or CD19 CAR.

In some embodiments, the markers of phenotype and activation of patient PBMCs are monitored by a panel comprising antibodies to CD3, CD8, CD45RA, CCR7, CD57, CD107a, CD279 and/or CD19 CAR.

In some embodiments, the markers of phenotype and activation of patient PBMCs are monitored by a panel comprising antibodies to CD3, CD8, CD45RA, CCR7, CD25, CD69, CD137 and/or CD19 CAR.

In some embodiments, the markers of phenotype and activation of patient PBMCs are monitored by a panel comprising antibodies to CD3, CD4, CD8, CD66b, CD19, CD14, CD56, and/or CD19 CAR.

In some embodiments, the markers are determined by a flow cytometry assay.

In one aspect, the present invention provides a CD19-directed genetically modified T-cell immunotherapy and a 4-1BB (CD137) agonist for use in a method of treating refractory diffuse large B-cell lymphoma in a patient in need thereof comprising: (a) administering to the patient a CD19-directed genetically modified T-cell immunotherapy; (b) administering to the patient a 4-1BB (CD137) agonist; and (c) monitoring the patient following administration for changes in markers of phenotype and activation of patient peripheral blood mononuclear cells (PBMCs).

In one aspect, the present invention provides a CD19-directed genetically modified T-cell immunotherapy and a 4-1BB (CD137) agonist for use in a method of treating refractory diffuse large B-cell lymphoma after two or more lines of systemic therapy in a patient in need thereof comprising: (a) administering to the patient a CD19-directed genetically modified T-cell immunotherapy; (b) administering to the patient a 4-1BB (CD137) agonist; and (c) monitoring the patient serum following administration for chemokine, cytokine and/or immune effector levels.

In one aspect, the present invention provides for a method of treating refractory diffuse large B-cell lymphoma in a patient in need thereof comprising: (a) administering to the patient a CD19-directed genetically modified T-cell immunotherapy; (b) administering to the patient a 4-1BB (CD137) agonist; and (c) monitoring the patient serum following administration for chemokine, cytokine and/or immune effector levels.

In one aspect, the present invention provides a method of treating refractory diffuse large B-cell lymphoma after two or more lines of systemic therapy in a patient in need thereof comprising: (a) administering to the patient a CD19-directed genetically modified T-cell immunotherapy; (b) administering to the patient a 4-1BB (CD137) agonist; and (c) monitoring the patient serum following administration for chemokine, cytokine and/or immune effector levels.

In some embodiments, the CD19-directed genetically modified T-cell immunotherapy is an autologous immunotherapy.

In some embodiments, the CD19-directed genetically modified T-cell immunotherapy is an allogenic immunotherapy.

In some embodiments, the T-cells are genetically modified ex vivo.

In some embodiments, the T-cells are genetically modified by retroviral transduction.

In some embodiments, the CD19-directed genetically modified T-cell immunotherapy, is genetically modified to express a chimeric antigen receptor (CAR) said CAR comprising an anti-CD19 single chain variable fragment (scFv) linked to CD28 and CD3-zeta co-stimulatory domains.

In some embodiments, the CD19-directed genetically modified T-cell immunotherapy is axicabtagene ciloleucel.

In some embodiments, the 4-1BB (CD137) agonist is an antigen binding molecule or fragment thereof.

In some embodiments, the 4-1BB (CD137) agonist is an isolated antibody, or antigen-binding portion thereof, comprising three CDRs of a VH region amino acid sequence as set forth in SEQ ID NO:1 and three CDRS of a VL region amino acid sequence set forth in SEQ ID NO: 3.

In some embodiments, the 4-1BB (CD137) agonist is an isolated antibody, or antigen-binding portion thereof, comprising: (a) a H-CDR1 as set forth in SEQ ID NO:5; (b) a H-CDR2 as set forth in SEQ ID NO:6; (c) a H-CDR3 as set forth in SEQ ID NO:7; (d) a L-CDR1 as set forth in SEQ ID NO:8; (e) a L-CDR2 as set forth in SEQ ID NO:9; and (f) a L-CDR3 as set forth in SEQ ID NO:10.

In some embodiments, the 4-1BB (CD137) agonist is a fully human monoclonal antibody.

In some embodiments, the 4-1BB (CD137) agonist comprises a VH region amino acid sequence as set forth in SEQ ID NO:1 and a VL region amino acid sequence as set forth in SEQ ID NO: 3.

In some embodiments, the 4-1BB (CD137) comprises a heavy chain amino acid sequence as set forth in SEQ ID NO:2 and a light chain amino acid sequence as set forth in SEQ ID NO:4, with the proviso that the C-terminal lysine residue of SEQ ID NO:2 is optionally absent.

In some embodiments, the 4-1BB (CD137) agonist is utomilumab.

In some embodiments, the patient serum is monitored for IL-15, IL-7, IL-2, IL-6, IL1α, IL-1β, IL-17α, TNFα, TNFβ, GM-CSF, CRP, SAA, IL-13, IL-4, IL-5, IL-10, IFNγ, IL-12p40, IL-12p70, IL-16, IL-8, MCP-1, MCP-4, MIP-1α, MIP-1β, IP-10, TARC, Eotaxin, Eotaxin-3, MDC, Granzyme A, Granzyme B, sFASL, Perforin, FGF-2, sICAM-1, sVCAM-1, VEGF, VEGF-C, VEGF-D, PLGF, IL1Rα, IL1Rβ, and/or Ferritin.

In some embodiments, the chemokine, cytokine and/or immune effector levels are determined using a multiplex assay.

In one aspect, the present invention provides a CD19-directed genetically modified T-cell immunotherapy and a 4-1BB (CD137) agonist for use in a method of treating refractory diffuse large B-cell lymphoma after two or more lines of systemic therapy in a patient in need thereof comprising: (a) administering to the patient a CD19-directed genetically modified T-cell immunotherapy; (b) administering to the patient a 4-1BB (CD137) agonist; and (c) monitoring the patient serum following administration for chemokine, cytokine and/or immune effector levels.

In one aspect, the present invention provides a method of treating refractory diffuse large B-cell lymphoma in a patient in need thereof comprising: (a) administering to the patient a CD19-directed genetically modified T-cell immunotherapy; (b) administering to the patient a 4-1BB (CD137) agonist; and (c) analyzing the patient response after administration for regression [complete response (CR) or partial response (PR)], refractory to treatment [progressive disease (PD)], relapse or persisting without evidence of progression or complete regression [prolonged PR or stable disease (SD)].

In one aspect, the present invention provides a method of treating refractory diffuse large B-cell lymphoma after two or more lines of systemic therapy in a patient in need thereof comprising: (a) administering to the patient a CD19-directed genetically modified T-cell immunotherapy; (b) administering to the patient a 4-1BB (CD137) agonist; and (c) analyzing the patient response after administration for regression [complete response (CR) or partial response (PR)], refractory to treatment [progressive disease (PD)], relapse or persisting without evidence of progression or complete regression [prolonged PR or stable disease (SD)].

In some embodiments, the CD19-directed genetically modified T-cell immunotherapy is an autologous immunotherapy.

In some embodiments, the CD19-directed genetically modified T-cell immunotherapy is an allogenic immunotherapy.

In some embodiments, the T-cells are genetically modified ex vivo.

In some embodiments, the T-cells are genetically modified by retroviral transduction.

In some embodiments, the CD19-directed genetically modified T-cell immunotherapy, is genetically modified to express a chimeric antigen receptor (CAR) said CAR comprising an anti-CD19 single chain variable fragment (scFv) linked to CD28 and CD3-zeta co-stimulatory domains.

In some embodiments, the CD19-directed genetically modified T-cell immunotherapy is axicabtagene ciloleucel.

In some embodiments, the 4-1BB (CD137) agonist is an antigen binding molecule or fragment thereof.

In some embodiments, the 4-1BB (CD137) agonist is an isolated antibody, or antigen-binding portion thereof, comprising three CDRs of a VH region amino acid sequence as set forth in SEQ ID NO:1 and three CDRS of a VL region amino acid sequence set forth in SEQ ID NO: 3.

In some embodiments, the 4-1BB (CD137) agonist is an isolated antibody, or antigen-binding portion thereof comprising: (a) a H-CDR1 as set forth in SEQ ID NO:5; (b) a H-CDR2 as set forth in SEQ ID NO:6; (c) a H-CDR3 as set forth in SEQ ID NO:7; (d) a L-CDR1 as set forth in SEQ ID NO:8; (e) a L-CDR2 as set forth in SEQ ID NO:9; and (f) a L-CDR3 as set forth in SEQ ID NO:10.

In some embodiments, the 4-1BB (CD137)) agonist is a fully human monoclonal antibody.

In some embodiments, the 4-1BB (CD137) agonist comprises a VH region amino acid sequence as set forth in SEQ ID NO:1 and a VL region amino acid sequence as set forth in SEQ ID NO: 3.

In some embodiments, the 4-1BB (CD137) agonist comprises a heavy chain amino acid sequence as set forth in SEQ ID NO:2 and a light chain amino acid sequence as set forth in SEQ ID NO:4, with the proviso that the C-terminal lysine residue of SEQ ID NO:2 is optionally absent.

In some embodiments, the 4-1BB (CD137) agonist is utomilumab.

In some embodiments, analyzing the patient response after administration for regression [complete response (CR) or partial response (PR)], refractory to treatment [progressive disease (PD)], relapse or persisting without evidence of progression or complete regression [prolonged PR or stable disease (SD)]comprises monitoring markers of phenotype and activation of patient PBMCs comprising a pan T-cell marker, cytotoxic T-cell marker, differentiation T-cell marker, differentiation marker, IL-2 receptor, activation marker, PD1, 4-1BB, helper T-cell marker, granulocyte marker, B-cell marker, monocyte/macrophage marker, NK cell marker, and/or axicabtagene ciloleucel identification.

In some embodiments, the markers of phenotype and activation of patient PBMCs are monitored by a panel comprising antibodies to CD3, CD8, CD45RA, CCR7, CD122, CD27, CD28, CD95, and/or CD19 CAR.

In some embodiments, the markers of phenotype and activation of patient PBMCs are monitored by a panel comprising antibodies to CD3, CD8, CD45RA, CCR7, CD57, CD107a, CD279 and/or CD19 CAR.

In some embodiments, the markers of phenotype and activation of patient PBMCs are monitored by a panel comprising antibodies to CD3, CD8, CD45RA, CCR7, CD25, CD69, CD137 and/or CD19 CAR.

In some embodiments, the markers of phenotype and activation of patient PBMCs are monitored by a panel comprising antibodies to CD3, CD4, CD8, CD66b, CD19, CD14, CD56, and/or CD19 CAR.

In some embodiments, the markers are determined by a flow cytometry assay.

In some embodiments, the patient serum is monitored for IL-15, IL-7, IL-2, IL-6, IL1α, IL-1β, IL-17α, TNFα, TNFβ, GM-CSF, CRP, SAA, IL-13, IL-4, IL-5, IL-10, IFNγ, IL-12p40, IL-12p70, IL-16, IL-8, MCP-1, MCP-4, MIP-1α, MIP-1β, IP-10, TARC, Eotaxin, Eotaxin-3, MDC, Granzyme A, Granzyme B, sFASL, Perforin, FGF-2, sICAM-1, sVCAM-1, VEGF, VEGF-C, VEGF-D, PLGF, IL1Rα, IL1Rβ, and/or Ferritin.

In some embodiments, the chemokine, cytokine and/or immune effector levels are determined using a multiplex assay.

In one aspect, the present invention provides a CD19-directed genetically modified T-cell immunotherapy and a 4-1BB (CD137) agonist for use in a method of treating refractory diffuse large B-cell lymphoma after two or more lines of systemic therapy in a patient in need thereof comprising: (a) administering to the patient a CD19-directed genetically modified T-cell immunotherapy; (b) administering to the patient a 4-1BB (CD137) agonist; and (c) analyzing the patient response after administration for complete response (CR), partial response (PR), stable disease (SD), or progressive disease (PD).

The present invention also provides a CD19-directed genetically modified T-cell immunotherapy and a 4-1BB (CD137) agonist for use in the disclosed methods of treatment; and also the use of a CD19-directed genetically modified T-cell immunotherapy and a 4-1BB (CD137) agonist in the manufacture of a medicament for us in the disclosed methods of treatment.

Other features and advantages of the disclosure will be apparent from the following detailed description, including the Examples, and the claims.

### BRIEF DESCRIPTION OF THE DRAWINGS

The patent or application file contains at least one drawing executed in color. Copies of this paper or patent application publication with color drawing(s) will be provided by the Office upon request and payment of the necessary fee. The drawings are for illustration purposes only, not for limitation.

FIG. 1. illustrates the study design evaluating the safety and efficacy of KTE-C19 (axicabtagene ciloleucel) in combination with utomilumab in subjects with refractory large B-cell lymphoma or refractory diffuse large B-cell lymphoma (DLBCL).

FIG. 2A-2C. depicts the relationship between anti-CD19 CAR levels in blood over the first 28 days post infusion (AUC₀₋₂₈) with A. objective response rate (ORR) (Complete remission (CR) or partial remission (PR)), B. (NE), and development of Grade ≥3 neurologic toxicity or C. cytokine release syndrome (CRS).

FIG. 3 Results of lymphodepletion chemotherapy and anti-CD19 CAR T induction of key immune programs over the first 28 days post infusion. Analytes shown were elevated in 50% of patients with ≥2-fold induction above baseline out of a panel of 44 measured. Serum analytes were measured MSD®, Luminex®, and Quantikine® ELISA. CRP, C-reactive protein; GM-CSF, granulocyte-macrophage colony-stimulating factor; IFN, interferon; IL, interleukin; MCP-1, monocyte chemoattractant protein-1; SAA, serum amyloid A.

FIG. 4 depicts biomarkers associated with both grade CRS and grade neurologic toxicity. The association between peak levels of serum analytes and association with Grade neurological toxicity or CRS are shown. Peak levels after Axi-cel™ infusion were used in the comparison. AUC, area under the curve; CRS, cytokine release syndrome; IFN, interferon; IL, interleukin, MCP, monocyte chemoattractant protein; NE, neurologic events.

FIG. 5A-5H Anti-CD19 CAR T-cells demonstrate broad polyfunctionality in co-culture with CD 19⁺ tumor cells. A-D. Cytokines: A. IL-2, B. IL-4, C. Granzyme B, D. IFNγ, E-H. Surface markers: E. CD69, F. CD107a, G. CD137, H. PD1.

FIG. 6 shows a proposed biopsy collection schedule to enable analysis of samples that fall into four general categories of response: 1) regression [complete response (CR) or partial response (PR)], 2) refractory to treatment [progressive disease (PD)], 3) relapse or 4) persisting without evidence of progression or complete regression [prolonged PR or stable disease (SD)].

FIG. 7 illustrates an exemplary biomarker sample collection schedule. Axi-cel: axicabtagene ciloleucel; CAR: chimeric antigen receptor; ELISA: enzyme-linked immunosorbent assay; qPCR: quantitative polymerase chain reaction.

FIG. 8 illustrates an exemplary paired biopsy collection schedule.

FIG. 9 illustrates sample processing schemes for core needle biopsies.

FIG. 10 depicts a schematic view of markers and analysis approaches to evaluate patient biopsy samples.

FIG. 11 shows the antibody sequence (Heavy Chain: SEQ ID NO: 2, Light Chain: SEQ ID NO: 4) and structural features of utomilumab.

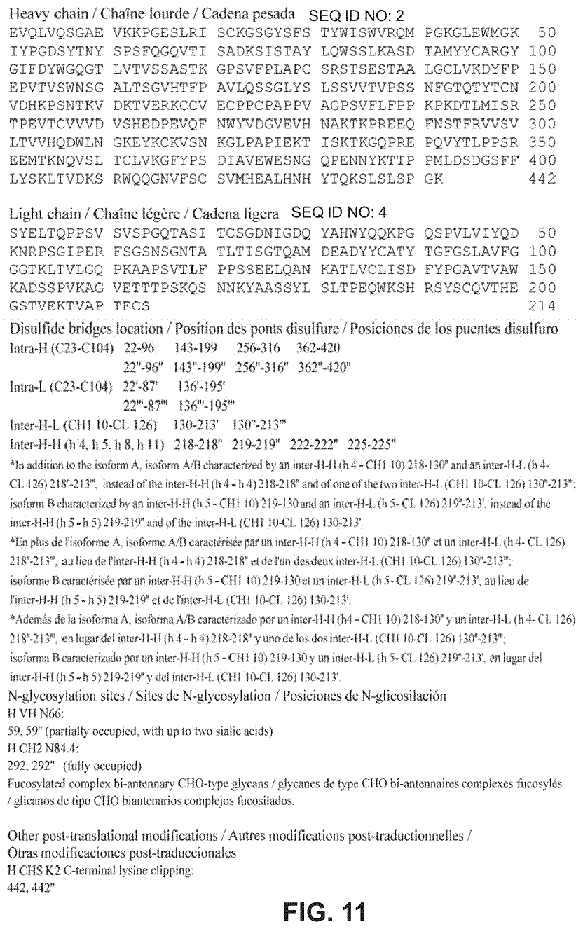

FIG. 12 depicts the mechanism of action of utomilumab.

FIG. 13 illustrates another study design evaluating the safety and efficacy of KTE-C19 (axicabtagene ciloleucel) in combination with utomilumab in subjects with refractory large B-cell lymphoma or refractory diffuse large B-cell lymphoma (DLBCL).

FIG. 14A-14B. The IL-2 production by the anti-CD19 CAR T-cells. The cells were incubated with the tool antibody (0.33 μg/mL) in the presence of the control antibody (A) or Utomilumab (B) over for 16 hours. The first data point on the X axis represents the tool antibody alone. Data represents average of triplicate wells.

### DETAILED DESCRIPTION

The present invention relates to a method of treating a disease or disorder in a patient comprising administering axicabtagene ciloleucel (KTE-C19) in combination with utomilumab (PF-05082566) a 4-1BB (CD137) agonist fully human IgG2 monoclonal antibody. Axicabtagene ciloleucel is a CD19-directed genetically modified autologous T-cell immunotherapy cell suspension, comprising the patient's own T-cells harvested and genetically modified ex vivo by retroviral transduction to express a chimeric antigen receptor (CAR) comprising an FMC63 anti-CD19 single chain variable fragment (scFv) linked to CD28 and CD3-zeta co-stimulatory domains. See e.g., Neelapu et al., Clin. Adv. Hem. Onc., Vol. 15, Issue 2 (2017). In some embodiments, the disease or disorder is lymphoma, such as refractory diffuse large B-cell lymphoma (DLBCL) or leukemia, such as acute lymphoblastic leukemia (ALL).

To prepare CD19-directed genetically modified autologous T-cell immunotherapy, a patient's own T-cells can be harvested and genetically modified ex vivo by retroviral transduction to express a chimeric antigen receptor (CAR) comprising a murine anti-CD19 single chain variable fragment (scFv) linked to CD28 and CD3-zeta co-stimulatory domains. In some embodiments, the CAR comprises a murine anti-CD19 single chain variable fragment (scFv) linked to 4-1BB and CD3-zeta co-stimulatory domain. The anti-CD19 CAR T-cells can be expanded and infused back into the patient, where they can recognize and eliminate CD19-expressing target cells. YESCARTA™ (Axi-cel™; axicabtagene ciloleucel) is an example of such CD19-directed genetically modified autologous T-cell immunotherapy. See Kochenderfer, et al., (J Immunother 2009; 32:689 702). Additional CD19 directed CAR therapies include JCAR017, JCAR015, JCAR014, Kymriah (tisagenlecleucel). See Sadelain et al., Nature Rev. Cancer Vol. 3 (2003), Ruella et al., Curr Hematol Malig Rep., Springer, N.Y. (2016) and Sadelain et al. Cancer Discovery (April 2013).

CD19-directed genetically modified autologous T-cell immunotherapy can be prepared from the patient's peripheral blood mononuclear cells, which are typically obtained via a standard leukapheresis procedure. The mononuclear cells can be enriched for T-cells and activated with anti-CD3 antibody in the presence of IL-2, then transduced with the replication incompetent retroviral vector containing the anti-CD19 CAR transgene. The transduced T-cells can be expanded in cell culture, washed, formulated into a suspension, and/or cryopreserved. Typically, the product comprising genetically modified autologous T-cells must pass a sterility test before release for shipping as a frozen suspension in a patient-specific infusion container such as an infusion bag. Typically, the product is thawed prior to infusion.

In addition to T-cells, CD19-directed genetically modified autologous T-cell immunotherapy may contain NK and NK-T cells. In some embodiments, the CD19-directed genetically modified autologous T-cell immunotherapy formulation contains about 5% dimethylsulfoxide (DMSO) and about 2.5% albumin (human) (v/v).

CD19-directed genetically modified autologous and/or allogeneic T-cells can bind to CD19-expressing cancer cells and normal B-cells. Certain studies have demonstrated that, following anti-CD19 CAR T-cell engagement with CD19-expressing target cells, the CD28 co-stimulatory and CD3-zeta activating domains trigger downstream signaling cascades that lead to T-cell activation, proliferation, acquisition of effector functions and secretion of inflammatory cytokines and chemokines. This sequence of events leads to killing of CD19-expressing cells.

The antigen-binding molecule or fragment thereof that binds 4-1BB and is suitable for the present invention is a 4-1BB antibody. In some embodiments, the antibody is a 4-1BB agonist antibody. In some embodiments, the 4-1BB (CD137) agonist is an isolated antibody, or antigen-binding portion thereof, comprising three CDRs of a VH region amino acid sequence as set forth in SEQ ID NO:1 and three CDRS of a VL region amino acid sequence set forth in SEQ ID NO: 3. In some embodiments, the antibody binds to human 4-1BB. In some embodiments the 4-1BB antibody comprises (a) a H-CDR1 as set forth in SEQ ID NO:5; (b) a H-CDR2 as set forth in SEQ ID NO:6; (c) a H-CDR3 as set forth in SEQ ID NO:7; (d) a L-CDR1 as set forth in SEQ ID NO:8; (e) a L-CDR2 as set forth in SEQ ID NO:9; and (f) a L-CDR3 as set forth in SEQ ID NO:10. In some embodiments, the CD137 (4-1BB) antibody comprises (1) a VH region amino acid sequence as set forth in SEQ ID NO:1, and (2) a VL region amino acid sequence as set forth in SEQ ID NO:3. In some embodiments, the 4-1BB antibody comprises a heavy chain amino acid sequence as set forth in SEQ ID NO:2 and a light chain amino acid sequence as set forth in SEQ ID NO:4, with the proviso that the C-terminal lysine residue of SEQ ID NO:2 is optionally absent. In some embodiments, the 4-1BB antibody is a fully human monoclonal antibody. Utomilumab is an example of such fully human monoclonal antibody that binds human 4-1BB.

Agonism of 4-1BB on engineered anti-CD19 CAR T cells may enhance antitumor activity of axicabtagene ciloleucel via the following mechanisms: (1) increasing the viability of anti-CD19 CAR T cells through upregulation of anti-apoptotic proteins, (2) enhancing anti-CD19 CAR T-cell expansion and proliferation, and 3) contributing to the T-cell immune response.

### Definitions

In order for the present invention to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.,” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” can mean within one or more than one standard deviation per the practice in the art. “About” or “approximately” can mean a range of up to 10% (i.e., ±10%). Thus, “about” can be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg can include any amount between 4.5 mg and 5.5 mg. Furthermore, particularly with respect to biological systems or processes, the terms can mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed., (2006), Oxford University Press, provide those of skill in the art with a general dictionary for many of the terms used in this disclosure.

“Administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “agonist” refers to a antigen binding molecule, as defined herein, which upon binding to 4-IBB, (1) stimulates or activates 4-IBB, (2) enhances, increases, promotes, induces, or prolongs an activity, function, or presence of 4-IBB, or (3) enhances, increases, promotes, or induces the expression of 4-IBB.

The term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding molecule thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region is comprises one constant domain, CL. The VH and VL regions can be further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system.

Antibodies can include, for example, monoclonal antibodies, recombinantly produced antibodies, monospecific antibodies, multispecific antibodies (including bispecific antibodies), human antibodies, engineered antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, antibody fusions (sometimes referred to herein as “antibody conjugates”), heteroconjugate antibodies, single domain antibodies, monovalent antibodies, single chain antibodies or single-chain Fvs (scFv), camelized antibodies, affybodies, Fab fragments, F(ab′)2 fragments, disulfide-linked Fvs (sdFv), anti-idiotypic (anti-Id) antibodies (including, e.g., anti-anti-Id antibodies), minibodies, domain antibodies, synthetic antibodies (sometimes referred to herein as “antibody mimetics”), and antigen-binding fragments of any of the above. In some embodiments, antibodies described herein refer to polyclonal antibody populations.

An “antigen binding molecule,” “antigen binding portion,” or “antibody fragment” refers to any molecule that comprises the antigen binding parts (e.g., CDRs) of the antibody from which the molecule is derived. An antigen binding molecule can include the antigenic complementarity determining regions (CDRs). Examples of antibody fragments include, but are not limited to, Fab, Fab′, F(ab′)2, and Fv fragments, dAb, linear antibodies, scFv antibodies, and multispecific antibodies formed from antigen binding molecules. Peptibodies (i.e., Fc fusion molecules comprising peptide binding domains) are another example of suitable antigen binding molecules. In some embodiments, the antigen binding molecule binds to an antigen on a tumor cell. In some embodiments, the antigen binding molecule binds to an antigen on a cell involved in a hyperproliferative disease or to a viral or bacterial antigen. In some embodiments, the antigen binding molecule binds to CD19. In some embodiments, the antigen binding molecule binds to 4-1BB (CD137). In further embodiments, the antigen binding molecule is an antibody fragment that specifically binds to the antigen, including one or more of the complementarity determining regions (CDRs) thereof. In further embodiments, the antigen binding molecule is a single chain variable fragment (scFv). In some embodiments, the antigen binding molecule comprises or consists of avimers.

An “antigen” refers to any molecule that provokes an immune response or is capable of being bound by an antibody or an antigen binding molecule. The immune response may involve either antibody production, or the activation of specific immunologically-competent cells, or both. A person of skill in the art would readily understand that any macromolecule, including virtually all proteins or peptides, can serve as an antigen. An antigen can be endogenously expressed, i.e., expressed by genomic DNA, or can be recombinantly expressed. An antigen can be specific to a certain tissue, such as a cancer cell, or it can be broadly expressed. In addition, fragments of larger molecules can act as antigens. In some embodiments, antigens are tumor antigens.

The term “antibody derivative” or “derivative” of an antibody refers to a molecule that is capable of binding to the same antigen (e.g., 4-1BB) that the antibody binds to and comprises an amino acid sequence of the antibody linked to an additional molecular entity. The amino acid sequence of the antibody that is contained in the antibody derivative may be a full-length heavy chain, a full-length light chain, any portion or portions of a full-length heavy chain, any portion or portions of the full-length light chain of the antibody, any other fragment(s) of an antibody, or the complete antibody. The additional molecular entity may be a chemical or biological molecule. Examples of additional molecular entities include chemical groups, amino acids, peptides, proteins (such as enzymes, antibodies), and chemical compounds. The additional molecular entity may have any utility, such as for use as a detection agent, label, marker, pharmaceutical or therapeutic agent. The amino acid sequence of an antibody may be attached or linked to the additional molecular entity by chemical coupling, genetic fusion, noncovalent association, or otherwise. The term “antibody derivative” also encompasses chimeric antibodies, humanized antibodies, and molecules that are derived from modifications of the amino acid sequences of a 4-1BB antibody, such as conservation amino acid substitutions, additions, and insertions.

The term “human antibody” refers to antibodies having variable and constant regions (if present) derived from human germline immunoglobulin sequences. The human antibodies of the present invention may include amino acid residues not encoded by human germline sequences (e.g., mutations introduced by random or site-specific mutagenesis in vitro or by somatic mutation in vivo). The term “human antibody” is not intended to include chimeric or humanized antibodies comprising non-human antigen binding residues.

“CD19-directed genetically modified autologous T-cell immunotherapy” refers to a suspension of chimeric antigen receptor (CAR)-positive T-cells. An example of such immunotherapy is axicabtagene ciloleucel (also known as Axi-cel™, YESCARTA™, developed by Kite Pharmaceuticals, Inc.

The term “neutralizing” refers to an antigen binding molecule, scFv, antibody, or a fragment thereof that binds to a ligand and prevents or reduces the biological effect of that ligand. In some embodiments, the antigen binding molecule, scFv, antibody, or a fragment thereof, directly blocking a binding site on the ligand or otherwise alters the ligand's ability to bind through indirect means (such as structural or energetic alterations in the ligand). In some embodiments, the antigen binding molecule, scFv, antibody, or a fragment thereof prevents the protein to which it is bound from performing a biological function.

The term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies may be prepared by the hybridoma methodology or may be made using recombinant DNA methods in bacterial, eukaryotic animal or plant cells (see, e.g., U.S. Pat. No. 4,816,567). Monoclonal antibodies may also be isolated from phage antibody libraries using the techniques described in Clackson et al., Nature, 352:624-628 (1991) and Marks et al., J. Mol. Biol., 222:581-597 (1991), for example.

The term “autologous” refers to any material derived from the same individual to which it is later to be re-introduced. For example, the engineered autologous cell therapy (eACT™) method described herein involves collection of lymphocytes from a patient, which are then engineered to express, e.g., a CAR construct, and then administered back to the same patient.

The term “allogeneic” refers to any material derived from one individual which is then introduced to another individual of the same species, e.g., allogeneic T-cell transplantation.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. A “cancer” or “cancer tissue” can include a tumor. Examples of cancers that can be treated by the methods disclosed herein include, but are not limited to, cancers of the immune system including lymphoma, leukemia, myeloma, and other leukocyte malignancies. In some embodiments, the methods disclosed herein can be used to reduce the tumor size of a tumor derived from, for example, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, multiple myeloma, Hodgkin's Disease, non-Hodgkin's lymphoma (NHL), primary mediastinal large B-cell lymphoma (PMBC), diffuse large B-cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T-cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, other B-cell malignancies, and combinations of said cancers. In some embodiments, the cancer is multiple myeloma. The particular cancer can be responsive to chemo- or radiation therapy or the cancer can be refractory. A refractor cancer refers to a cancer that is not amendable to surgical intervention and the cancer is either initially unresponsive to chemo- or radiation therapy or the cancer becomes unresponsive over time.

A “tumor” as used herein, refers to an abnormal mass of tissue that results when cells divide more than they should or do not die when they should. Tumors may be benign (not cancerous), or malignant (cancer). Tumors are also referred to as “neoplasms”. A “solid tumor” is an abnormal mass of tissue that usually does not contain cysts or liquid areas. Solid tumors may be benign (not cancerous), or malignant (cancer). Different types of solid tumors are named for the type of cells that form them. Examples of solid tumors are sarcomas, and carcinomas. Conversely, “liquid tumors”, e.g. lymphomas and leukemias (also referred to as cancers of the blood) generally do not form solid tumors.

An “anti-tumor effect” as used herein, refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor. An anti-tumor effect can also refer to the prevention of the occurrence of a tumor, e.g., a vaccine.

A “cytokine,” as used herein, refers to a non-antibody protein that is released by one cell in response to contact with a specific antigen, wherein the cytokine interacts with a second cell to mediate a response in the second cell. “Cytokine” as used herein is meant to refer to proteins released by one cell population that act on another cell as intercellular mediators. A cytokine can be endogenously expressed by a cell or administered to a subject. Cytokines may be released by immune cells, including macrophages, B-cells, T-cells, and mast cells to propagate an immune response. Cytokines can induce various responses in the recipient cell. Cytokines can include homeostatic cytokines, chemokines, pro-inflammatory cytokines, effectors, and acute-phase proteins. For example, homeostatic cytokines, including interleukin (IL) 7 and IL-15, promote immune cell survival and proliferation, and pro-inflammatory cytokines can promote an inflammatory response. Examples of homeostatic cytokines include, but are not limited to, IL-2, IL-4, IL-5, IL-7, IL-10, IL-12p40, IL-12p70, IL-15, and interferon (IFN) gamma. Examples of pro-inflammatory cytokines include, but are not limited to, IL-1a, IL-1b, IL-6, IL-13, IL-17a, tumor necrosis factor (TNF)-alpha, TNF-beta, fibroblast growth factor (FGF) 2, granulocyte macrophage colony-stimulating factor (GM-CSF), soluble intercellular adhesion molecule 1 (sICAM-1), soluble vascular adhesion molecule 1 (sVCAM-1), vascular endothelial growth factor (VEGF), VEGF-C, VEGF-D, and placental growth factor (PLGF). Examples of effectors include, but are not limited to, granzyme A, granzyme B, soluble Fas ligand (sFasL), and perforin. Examples of acute phase-proteins include, but are not limited to, C-reactive protein (CRP) and serum amyloid A (SAA).

“Chemokines” are a type of cytokine that mediates cell chemotaxis, or directional movement. Examples of chemokines include, but are not limited to, IL-8, IL-16, eotaxin, eotaxin-3, macrophage-derived chemokine (MDC or CCL22), monocyte chemotactic protein 1 (MCP-1 or CCL2), MCP-4, macrophage inflammatory protein 1α (MIP-1α, MIP-1α), MIP-1β (MIP-1b), gamma-induced protein 10 (IP-10), and thymus and activation regulated chemokine (TARC or CCL17).

A “therapeutically effective amount,” “effective dose,” “effective amount,” or “therapeutically effective dosage” of a therapeutic agent, e.g., engineered CAR T-cells, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The term “lymphocyte” as used herein includes natural killer (NK) cells, T cells, or B-cells. NK cells are a type of cytotoxic (cell toxic) lymphocyte that represent a major component of the inherent immune system. NK cells reject tumors and cells infected by viruses. It works through the process of apoptosis or programmed cell death. They were termed “natural killers” because they do not require activation in order to kill cells. T cells play a major role in cell-mediated-immunity (no antibody involvement). Their T-cell receptors (TCRs) differentiate themselves from other lymphocyte types. The thymus, a specialized organ of the immune system, is primarily responsible for the T-cell's maturation. There are six types of T-cells, namely: Helper T-cells (e.g., CD4+ cells), Cytotoxic T-cells (also known as a TC, cytotoxic T lymphocyte, CTL, T-killer cell, cytolytic T-cell, CD8+ T-cells or killer T-cell), Memory T-cells ((i) stem memory TSCM cells, like naive cells, are CD45RO−, CCR7+, CD45RA+, CD62L+(L-selectin), CD27+, CD28+ and IL-7Rα+, but they also express large amounts of CD95, IL-2Rβ, CXCR3, and LFA-1, and show numerous functional attributes distinctive of memory cells); (ii) central memory TCM cells express L-selectin and the CCR7, they secrete IL-2, but not IFNγ or IL-4, and (iii) effector memory TEM cells, however, do not express L-selectin or CCR7 but produce effector cytokines like IFNγ and IL-4), Regulatory T-cells (Tregs, suppressor T-cells, or CD4+CD25+ regulatory T-cells), Natural Killer T-cells (NKT) and Gamma Delta T-cells. B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

The term “genetically modified”, “genetically engineered” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T-cell, which can either be obtained from a patient or a donor. The cell can be modified to express an exogenous construct, such as, e.g., a chimeric antigen receptor (CAR) or a T-cell receptor (TCR), which is incorporated into the cell's genome.

An “immune response” refers to the action of a cell of the immune system (for example, T lymphocytes, B lymphocytes, natural killer (NK) cells, macrophages, eosinophils, mast cells, dendritic cells and neutrophils) and soluble macromolecules produced by any of these cells or the liver (including Abs, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “selectively binds” or “selectively binds to,” or “selective targeting” in reference to the interaction of a binding molecule, as defined herein, (e.g., an antibody) with its binding partner (e.g., an antigen), refers to the ability of the binding molecule to discriminate between an antigen of interest from an animal species (such as human 4-1BB) and a different antigen from the same animal species (such as human CD40) under a given set of conditions. A 4-1BB binding molecule is said to selectively bind to human 4-1BB if it binds to human 4-1BB at an EC50 that is below 10 percent of the EC50 at which it binds to human CD40 or human CD134 as determined in an in vitro assay.

The term “immunotherapy” refers to the treatment of a subject afflicted with, or at risk of contracting or suffering a recurrence of, a disease by a method comprising inducing, enhancing, suppressing or otherwise modifying an immune response. Examples of immunotherapy include, but are not limited to, T-cell therapies. T-cell therapy can include adoptive T-cell therapy, tumor-infiltrating lymphocyte (TIL) immunotherapy, autologous cell therapy, engineered autologous cell therapy (eACT™), and allogeneic T-cell transplantation. However, one of skill in the art would recognize that the conditioning methods disclosed herein would enhance the effectiveness of any transplanted T-cell therapy. Examples of T-cell therapies are described in U.S. Patent Publication Nos. 2014/0154228 and 2002/0006409, U.S. Pat. Nos. 7,741,465, 6,319,494, 5,728,388, and PCT Publication No. WO 2008/081035.

The T-cells of the immunotherapy can come from any source known in the art. For example, T-cells can be differentiated in vitro from a hematopoietic stem cell population, or T-cells can be obtained from a subject. T-cells can be obtained from, e.g., peripheral blood mononuclear cells (PBMCs), bone marrow, lymph node tissue, cord blood, thymus tissue, tissue from a site of infection, ascites, pleural effusion, spleen tissue, and tumors. In addition, the T-cells can be derived from one or more T-cell lines available in the art. T-cells can also be obtained from a unit of blood collected from a subject using any number of techniques known to the skilled artisan, such as FICOLL™ separation and/or apheresis. Additional methods of isolating T-cells for a T-cell therapy are disclosed in U.S. Patent Publication No. 2013/0287748, which is herein incorporated by references in its entirety.

The term “engineered Autologous Cell Therapy,” which can be abbreviated as “eACT™,” also known as adoptive cell transfer, is a process by which a patient's own T-cells are collected and subsequently genetically altered to recognize and target one or more antigens expressed on the cell surface of one or more specific tumor cells or malignancies. T-cells can be engineered to express, for example, chimeric antigen receptors (CAR). CAR positive (+) T-cells are engineered to express an extracellular single chain variable fragment (scFv) with specificity for a particular tumor antigen linked to an intracellular signaling part comprising at least one costimulatory domain and at least one activating domain. The CAR scFv can be designed to target, for example, CD19, which is a transmembrane protein expressed by cells in the B-cell lineage, including all normal B-cells and B-cell malignances, including but not limited to diffuse large B-cell lymphoma (DLBCL) not otherwise specified, primary mediastinal large B-cell lymphoma, high grade B-cell lymphoma, and DLBCL arising from follicular lymphoma, NHL, CLL, and non-T-cell ALL. Example CAR T-cell therapies and constructs are described in U.S. Patent Publication Nos. 2013/0287748, 2014/0227237, 2014/0099309, and 2014/0050708, and these references are incorporated by reference in their entirety.

A “patient” as used herein includes any human who is afflicted with a cancer (e.g., a lymphoma or a leukemia). The terms “subject” and “patient” are used interchangeably herein.

As used herein, the term “in vitro cell” refers to any cell which is cultured ex vivo. In particular, an in vitro cell can include a T-cell.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

“Stimulation,” as used herein, refers to a primary response induced by binding of a stimulatory molecule with its cognate ligand, wherein the binding mediates a signal transduction event. A “stimulatory molecule” is a molecule on a T-cell (e.g., the T-cell receptor (TCR)/CD3 complex) that specifically binds with a cognate stimulatory ligand present on an antigen present cell. A “stimulatory ligand” is a ligand that when present on an antigen presenting cell (e.g., an APC, a dendritic cell, a B-cell, and the like) can specifically bind with a stimulatory molecule on a T-cell, thereby mediating a primary response by the T-cell, including, but not limited to, activation, initiation of an immune response, proliferation, and the like. Stimulatory ligands include, but are not limited to, an anti-CD3 antibody, an MHC Class I molecule loaded with a peptide, a superagonist anti-CD2 antibody, and a superagonist anti-CD28 antibody.

A “costimulatory signal,” as used herein, refers to a signal, which in combination with a primary signal (Signal 1), such as TCR/CD3 ligation and/or activation, leads to a T-cell response, such as, but not limited to, proliferation and/or upregulation or down regulation of key molecules.

A “costimulatory ligand,” as used herein, includes a molecule on an antigen presenting cell that specifically binds a cognate co-stimulatory molecule on a T-cell. Binding of the costimulatory ligand provides a signal that mediates a T-cell response, including, but not limited to, proliferation, activation, differentiation, and the like. A costimulatory ligand induces a signal that is in addition to the primary signal provided by a stimulatory molecule, for instance, by binding of a T-cell receptor (TCR)/CD3 complex with a major histocompatibility complex (MHC) molecule loaded with peptide. A co-stimulatory ligand can include, but is not limited to, 3/TR6, 4-1BB ligand, agonist or antibody that binds Toll ligand receptor, B7-1 (CD80), B7-2 (CD86), CD30 ligand, CD40, CD7, CD70, CD83, herpes virus entry mediator (HVEM), human leukocyte antigen G (HLA-G), ILT4, immunoglobulin-like transcript (ILT) 3, inducible costimulatory ligand (ICOS-L), intercellular adhesion molecule (ICAM), ligand that specifically binds with B7-H3, lymphotoxin beta receptor, MHC class I chain-related protein A (MICA), MHC class I chain-related protein B (MICB), OX40 ligand, PD-L2, or programmed death (PD) L1. A co-stimulatory ligand includes, without limitation, an antibody that specifically binds with a co-stimulatory molecule present on a T-cell, such as, but not limited to, 4-1BB, B7-H3, CD2, CD27, CD28, CD30, CD40, CD7, ICOS, ligand that specifically binds with CD83, lymphocyte function-associated antigen-1 (LFA-1), natural killer cell receptor C (NKG2C), OX40, PD-1, or tumor necrosis factor superfamily member 14 (TNFSF14 or LIGHT).

A “costimulatory molecule” is capable of mediating a costimulatory response by the T-cell, such as, but not limited to, proliferation. Costimulation is often referred to as “Signal 2.” Costimulatory molecules include, but are not limited to, 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CDI-la, CDI-lb, CDI-lc, CDI-ld, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGBI, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CDI la/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Lyl08), SLAMF7, SLP-76, TNF, TNFr, TNFR2, Toll ligand receptor, TRANCE/RANKL, VLA1, or VLA-6, or fragments, truncations, or combinations thereof.

A “costimulatory domain” as used herein refers to all or a portion (fragment, truncations) or combinations thereof of a costimulatory molecule engineered into a CAR. In some embodiments, a costimulatory domain is derived from 4-1BB/CD137, B7-H3, BAFFR, BLAME (SLAMF8), BTLA, CD 33, CD 45, CD100 (SEMA4D), CD103, CD134, CD137, CD154, CD16, CD160 (BY55), CD18, CD19, CD19a, CD2, CD22, CD247, CD27, CD276 (B7-H3), CD28, CD29, CD3 (alpha; beta; delta; epsilon; gamma; zeta), CD30, CD37, CD4, CD4, CD40, CD49a, CD49D, CD49f, CD5, CD64, CD69, CD7, CD80, CD83 ligand, CD84, CD86, CD8alpha, CD8beta, CD9, CD96 (Tactile), CDI-la, CDI-lb, CDI-lc, CDI-Id, CDS, CEACAM1, CRT AM, DAP-10, DNAM1 (CD226), Fc gamma receptor, GADS, GITR, HVEM (LIGHTR), IA4, ICAM-1, ICAM-1, ICOS, Ig alpha (CD79a), IL2R beta, IL2R gamma, IL7R alpha, integrin, ITGA4, ITGA4, ITGA6, ITGAD, ITGAE, ITGAL, ITGAM, ITGAX, ITGB2, ITGB7, ITGBI, KIRDS2, LAT, LFA-1, LFA-1, LIGHT, LIGHT (tumor necrosis factor superfamily member 14; TNFSF14), LTBR, Ly9 (CD229), lymphocyte function-associated antigen-1 (LFA-1 (CDI la/CD18), MHC class I molecule, NKG2C, NKG2D, NKp30, NKp44, NKp46, NKp80 (KLRF1), OX40, PAG/Cbp, PD-1, PSGL1, SELPLG (CD162), signaling lymphocytic activation molecule, SLAM (SLAMF1; CD150; IPO-3), SLAMF4 (CD244; 2B4), SLAMF6 (NTB-A; Lyl08), SLAMF7, SLP-76, TNF, TNFr, TNFR2, Toll ligand receptor, TRANCE/RANKL, VLA1, or VLA-6, or fragments, truncations, or combinations thereof. In some embodiments, a costimulatory domain is derived from CD28, 4-1BB, CD8, CD16, ICOS.

The terms “reducing” and “decreasing” are used interchangeably herein and indicate any change that is less than the original. “Reducing” and “decreasing” are relative terms, requiring a comparison between pre- and post-measurements. “Reducing” and “decreasing” include complete depletions.

“Treatment” or “treating” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease. In some embodiments, “treatment” or “treating” includes a partial remission. In another embodiment, “treatment” or “treating” includes a complete remission.

The terms “4-IBB antibody”, “CD137 antibody” and “4-1BB (CD137) antibody” are used interchangeable and refer to an antibody, as defined herein, capable of binding to human 4-IBB (CD137) receptor. The terms “4-IBB” and “4-IBB receptor” are used interchangeably in the present application, and include the human 4-IBB receptor, as well as variants, isoforms, and species homologs thereof. Accordingly, a binding molecule, as defined and disclosed herein, may also bind 4-IBB from species other than human. In other cases, a binding molecule may be completely specific for the human 4-1BB and may not exhibit species or other types of cross-reactivity. An example of such 4-1BB antibody is utomilumab (PF-05082566) developed by Pfizer Inc. Another example of a 4-1BB antibody is urelumab (BMS-663513).

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

Various aspects of the disclosure are described in further detail in the following subsections.

### Chimeric Antigen Receptors

Chimeric antigen receptors (CARs or CAR-Ts) are genetically engineered receptors. These engineered receptors can be readily inserted into and expressed by immune cells, including T-cells in accordance with techniques known in the art. With a CAR, a single receptor can be programmed to both recognize a specific antigen and, when bound to that antigen, activate the immune cell to attack and destroy the cell bearing that antigen. When these antigens exist on tumor cells, an immune cell that expresses the CAR can target and kill the tumor cell.

### Engineered T-Cells and Use

A CD19-directed genetically modified T-cell immunotherapy for the treatment of patients with relapsed or refractory large B-cell lymphoma after two or more lines of systemic therapy, including diffuse large B-cell lymphoma (DLBCL) not otherwise specified, primary mediastinal large B-cell lymphoma, high grade B-cell lymphoma, and DLBCL arising from follicular lymphoma is described herein. In some embodiments, the CD19-directed immunotherapy is autologous. In some embodiments, the CD19-directed immunotherapy is allogenic. In some embodiments, the CD19-directed genetically modified autologous T-cell immunotherapy is axicabtagene ciloleucel (Axi-cel™, YESCARTA™).

The cell of the present disclosure may be obtained through T-cells obtained from a subject. T-cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T-cells can be derived from one or more T-cell lines available in the art. T-cells can also be obtained from a unit of blood collected from a subject using any number of techniques known to the skilled artisan, such as FICOLL™ separation and/or apheresis. In some embodiments, the cells collected by apheresis are washed to remove the plasma fraction, and placed in an appropriate buffer or media for subsequent processing. In some embodiments, the cells are washed with PBS. As will be appreciated, a washing step can be used, such as by using a semiautomated flow through centrifuge, e.g., the Cobe™ 2991 cell processor, the Baxter CytoMate™, or the like. In some embodiments, the washed cells are resuspended in one or more biocompatible buffers, or other saline solution with or without buffer. In some embodiments, the undesired components of the apheresis sample are removed. Additional methods of isolating T-cells for a T-cell therapy are disclosed in U.S. Patent Pub. No. 2013/0287748, which is herein incorporated by references in its entirety.

In some embodiments, T-cells are isolated from PBMCs by lysing the red blood cells and depleting the monocytes, e.g., by using centrifugation through a PERCOLL™ gradient. In some embodiments, a specific subpopulation of T-cells, such as CD4+, CD8+, CD28+, CD45RA+, and CD45RO+ T-cells is further isolated by positive or negative selection techniques known in the art. For example, enrichment of a T-cell population by negative selection can be accomplished with a combination of antibodies directed to surface markers unique to the negatively selected cells. In some embodiments, cell sorting and/or selection via negative magnetic immunoadherence or flow cytometry that uses a cocktail of monoclonal antibodies directed to cell surface markers present on the cells negatively selected can be used. For example, to enrich for CD4+ cells by negative selection, a monoclonal antibody cocktail typically includes antibodies to CD8, CD11b, CD14, CD16, CD20, and HLA-DR. In some embodiments, flow cytometry and cell sorting are used to isolate cell populations of interest for use in the present disclosure.

In some embodiments, PBMCs are used directly for genetic modification with the immune cells (such as CARs) using methods as described herein. In some embodiments, after isolating the PBMCs, T lymphocytes are further isolated, and both cytotoxic and helper T lymphocytes are sorted into naive, memory, and effector T-cell subpopulations either before or after genetic modification and/or expansion.

In some embodiments, CD8+ cells are further sorted into naive, central memory, and effector cells by identifying cell surface antigens that are associated with each of these types of CD8+ cells. In some embodiments, the expression of phenotypic markers of central memory T-cells includes CCR7, CD3, CD28, CD45RO, CD62L, and CD127 and are negative for granzyme B. In some embodiments, central memory T-cells are CD8+, CD45RO+, and CD62L+ T-cells. In some embodiments, effector T-cells are negative for CCR7, CD28, CD62L, and CD127 and positive for granzyme B and perforin. In some embodiments, CD4+ T-cells are further sorted into subpopulations. For example, CD4+T helper cells can be sorted into naive, central memory, and effector cells by identifying cell populations that have cell surface antigens.

A suitable sequence for use in accordance with the invention can be found at GenBank deposit no. HM852952.1 (https://www.ncbi.nlm.nih.gov/nuccore/305690546). Additionally, methods for producing and/or manufacturing T cells expressing chimeric antigen receptors have been described in, e.g., PCT Publication No. WO2015120096, the contents of which are hereby incorporated by reference in their entirety. In some embodiments, the immune cells, e.g., T-cells, are genetically modified following isolation using known methods, or the immune cells are activated and expanded (or differentiated in the case of progenitors) in vitro prior to being genetically modified. In another embodiment, the immune cells, e.g., T-cells, are genetically modified with the chimeric antigen receptors described herein (e.g., transduced with a viral vector comprising one or more nucleotide sequences encoding a CAR) and then are activated and/or expanded in vitro. Further methods for activating and expanding T-cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. Generally, such methods include contacting PBMC or isolated T-cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T-cells. In other embodiments, the T-cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In some embodiments, the T-cells are obtained from a donor subject. In some embodiments, the donor subject is human patient afflicted with a cancer or a tumor. In some embodiments, the donor subject is a human patient not afflicted with a cancer or a tumor.

In some embodiments, the composition comprises a pharmaceutically acceptable carrier, diluent, solubilizer, emulsifier, preservative and/or adjuvant. In some embodiments, the composition comprises an excipient.

In some embodiments, the composition is selected for parenteral delivery, for inhalation, or for delivery through the digestive tract, such as orally. The preparation of such pharmaceutically acceptable compositions is within the ability of one skilled in the art. In some embodiments, buffers are used to maintain the composition at physiological pH or at a slightly lower pH, typically within a pH range of from about 5 to about 8. In some embodiments, when parenteral administration is contemplated, the composition is in the form of a pyrogen-free, parenterally acceptable aqueous solution comprising a composition described herein, with or without additional therapeutic agents, in a pharmaceutically acceptable vehicle. In some embodiments, the vehicle for parenteral injection is sterile distilled water in which composition described herein, with or without at least one additional therapeutic agent, is formulated as a sterile, isotonic solution, properly preserved. In some embodiments, the preparation involves the formulation of the desired molecule with polymeric compounds (such as polylactic acid or polyglycolic acid), beads or liposomes, that provide for the controlled or sustained release of the product, which are then be delivered via a depot injection. In some embodiments, implantable drug delivery devices are used to introduce the desired molecule.

In some embodiments, the methods of treating a cancer in a subject in need thereof comprise a T-cell therapy. In some embodiments, the T-cell therapy disclosed herein is engineered Autologous Cell Therapy (eACT™). According to this embodiment, the method can include collecting blood cells from the patient. The isolated blood cells (e.g., T-cells) can then be engineered to express a CAR or a TCR disclosed herein. In a particular embodiment, the CAR T-cells or the TCR T-cells are administered to the patient. In some embodiments, the CAR T-cells or the TCR T-cells treat a tumor or a cancer in the patient. In some embodiments the CAR T-cells or the TCR T-cells reduce the size of a tumor or a cancer.

In some embodiments, the donor T-cells for use in the T-cell therapy are obtained from the patient (e.g., for an autologous T-cell therapy). In other embodiments, the donor T-cells for use in the T-cell therapy are obtained from a subject that is not the patient. The T-cells can be administered at a therapeutically effective amount. For example, a therapeutically effective amount of the T-cells can be at least about 10⁴ cells, at least about 10⁵ cells, at least about 10⁶ cells, at least about 10⁷ cells, at least about 10⁸ cells, at least about 10⁹, or at least about 10¹⁰. In another embodiment, the therapeutically effective amount of the T-cells is about 10⁴ cells, about 10⁵ cells, about 10⁶ cells, about 10⁷ cells, or about 10⁸ cells. In some embodiments, the therapeutically effective amount of the CAR T-cells is about 2×10⁶ cells/kg, about 3×10⁶ cells/kg, about 4×10⁶ cells/kg, about 5×10⁶ cells/kg, about 6×10⁶ cells/kg, about 7×10⁶ cells/kg, about 8×10⁶ cells/kg, about 9×10⁶ cells/kg, about 1×10⁷ cells/kg, about 2×10⁷ cells/kg, about 3×10⁷ cells/kg, about 4×10⁷ cells/kg, about 5×10⁷ cells/kg, about 6×10⁷ cells/kg, about 7×10⁷ cells/kg, about 8×10⁷ cells/kg, or about 9×10⁷ cells/kg. In some embodiments, the therapeutically effective amount of the CAR-positive viable T-cells is between about 1×10⁶ and about 2×10⁶ CAR-positive viable T-cells per kg body weight up to a maximum dose of about 1×10⁸ CAR-positive viable T-cells.

### Methods of Treatment

The methods disclosed herein can be used to treat a cancer in a subject, reduce the size of a tumor, kill tumor cells, prevent tumor cell proliferation, prevent growth of a tumor, eliminate a tumor from a patient, prevent relapse of a tumor, prevent tumor metastasis, induce remission in a patient, or any combination thereof. In some embodiments, the methods induce a complete response. In other embodiments, the methods induce a partial response.

The present invention also provides a CD19-directed genetically modified T-cell immunotherapy and a 4-1BB (CD137) agonist for use in the disclosed methods of treatment; and also the use of a CD19-directed genetically modified T-cell immunotherapy and a 4-1BB (CD137) agonist in the manufacture of a medicament for us in the disclosed methods of treatment.

Cancers that may be treated include tumors that are not vascularized, not yet substantially vascularized, or vascularized. The cancer may also include solid or non-solid tumors. In some embodiments, the cancer is a hematologic cancer. In some embodiments, the cancer is of the white blood cells. In other embodiments, the cancer is of the plasma cells. In some embodiments, the cancer is leukemia, lymphoma, or myeloma. In some embodiments, the cancer is adult and/or pediatric acute lymphoblastic leukemia (ALL) (including non T-cell ALL), AIDS-related lymphoma, ALK-positive large B-cell lymphoma, acute lymphoid leukemia (ALL), and hemophagocytic lymphohistocytosis (HLH)), B-cell prolymphocytic leukemia, B-cell acute lymphoid leukemia (“BALL”), blastic plasmacytoid dendritic cell neoplasm, Burkitt's lymphoma, chronic lymphocytic leukemia (CLL), chronic myelogenous leukemia (CML), chronic myeloid leukemia (CML), chronic or acute granulomatous disease, chronic or acute leukemia, Classical Hodgkin lymphoma, refractory diffuse large B-cell lymphoma, diffuse large B-cell lymphoma (DLBCL), follicular lymphoma, follicular lymphoma (FL), hairy cell leukemia, hemophagocytic syndrome (Macrophage Activating Syndrome (MAS), Hodgkin's Disease, Intravascular large B-cell lymphoma, large cell granuloma, Large B-cell lymphoma arising in HHV8-associated multicentric Castleman's disease, Lymphomatoid granulomatosis, Lymphoplasmacytic lymphoma, leukocyte adhesion deficiency, malignant lymphoproliferative conditions, Mucosa-Associated Lymphatic Tissue lymphoma (MALT), mantle cell lymphoma (MCL), Marginal zone lymphoma (MZL), monoclonal gammapathy of undetermined significance (MGUS), multiple myeloma, myelodysplasia and myelodysplastic syndrome (MDS), myeloid diseases including but not limited to acute myeloid leukemia (AML), Nodal marginal zone B-cell lymphoma (NMZL), Nodular lymphocyte predominant Hodgkin's lymphoma, non-Hodgkin's lymphoma (NHL), plasma cell proliferative disorders (e.g., asymptomatic myeloma (smoldering multiple myeloma or indolent myeloma), plasmablastic lymphoma, plasmacytoid dendritic cell neoplasm, plasmacytomas (e.g., plasma cell dyscrasia; solitary myeloma; solitary plasmacytoma; extramedullary plasmacytoma; and multiple plasmacytoma), POEMS syndrome (Crow-Fukase syndrome; Takatsuki disease; PEP syndrome), Primary central nervous system lymphoma, Primary effusion lymphoma, primary mediastinal large B-cell lymphoma (PMBCL), small cell- or a large cell-follicular lymphoma, splenic marginal zone lymphoma (SMZL), systemic amyloid light chain amyloidosis, T-cell acute lymphoid leukemia (“TALL”), T-cell lymphoma, transformed follicular lymphoma, Waldenstrom macroglobulinemia, or a combination thereof.

In some embodiments, the cancer is Acute Lymphoblastic Leukemia (ALL), AIDS-related lymphoma, ALK-positive large B-cell lymphoma, Burkitt's lymphoma, Chronic lymphocytic leukemia, CLL), Classical Hodgkin lymphoma, Diffuse large B-cell lymphoma (DLBCL), Primary Mediastinal Large B-cell Lymphoma (PMBCL), Follicular lymphoma, Intravascular large B-cell lymphoma, Large B-cell lymphoma arising in HHV8-associated multicentric Castleman's disease, Lymphomatoid granulomatosis, Lymphoplasmacytic lymphoma, Mantle cell lymphoma (MCL), Marginal zone B-cell lymphoma (MZL), Mucosa-Associated Lymphatic Tissue lymphoma (MALT), Nodal marginal zone B-cell lymphoma (NMZL), Nodular lymphocyte predominant Hodgkin's lymphoma, Non-Hodgkin's lymphoma, Plasmablastic lymphoma, Primary central nervous system lymphoma, Primary effusion lymphoma, Splenic marginal zone lymphoma (SMZL), and Waldenström's macroglobulinemia, relapsed or refractory large B-cell lymphoma, diffuse large B-cell lymphoma (DLBCL) not otherwise specified, high grade B-cell lymphoma, and DLBCL arising from follicular lymphoma.

In some embodiments, the cancer is a myeloma. In some embodiments, the cancer is multiple myeloma. In some embodiments, the cancer is a leukemia. In some embodiments, the cancer is acute myeloid leukemia.

Binding molecules and pharmaceutical compositions provided by the present disclosure are useful for therapeutic, diagnostic, or other purposes, such as enhancing an immune response, treating cancer, enhancing efficacy of combination therapies, enhancing vaccine efficacy, or treating autoimmune diseases. In some aspects, the present disclosure provides a method of treating a disorder in a mammal, which comprises administering to the human in need of treatment a therapeutically effective amount of a CD19-directed genetically modified T-cell immunotherapy and a 4-1BB agonist.

In some embodiments, the disorder is a cancer. A variety of cancers where 4-1BB is implicated, whether malignant or benign and whether primary or secondary, may be treated or prevented with a method provided by the disclosure. Examples of such cancers include lung cancers such as bronchogenic carcinoma (e.g., squamous cell carcinoma, small cell carcinoma, large cell carcinoma, and adenocarcinoma), alveolar cell carcinoma, bronchial adenoma, chondromatous hamartoma (noncancerous), and sarcoma (cancerous); heart cancer such as myxoma, fibromas, and rhabdomyomas; bone cancers such as osteochondromas, condromas, chondroblastomas, chondromyxoid fibromas, osteoid osteomas, giant cell tumors, chondrosarcoma, multiple myeloma, osteosarcoma, fibrosarcomas, malignant fibrous histiocytomas, Ewing's tumor (Ewing's sarcoma), and reticulum cell sarcoma; brain cancer such as gliomas (e.g., glioblastoma multiforme), anaplastic astrocytomas, astrocytomas, oligodendrogliomas, medulloblastomas, chordoma, Schwannomas, ependymomas, meningiomas, pituitary adenoma, pinealoma, osteomas, hemangioblastomas, craniopharyngiomas, chordomas, germinomas, teratomas, dermoid cysts, and angiomas; cancers in digestive system such as leiomyoma, epidermoid carcinoma, adenocarcinoma, leiomyosarcoma, stomach adenocarcinomas, intestinal lipomas, intestinal neurofibromas, intestinal fibromas, polyps in large intestine, and colorectal cancers; liver cancers such as hepatocellular adenomas, hemangioma, hepatocellular carcinoma, fibrolamellar carcinoma, cholangiocarcinoma, hepatoblastoma, and angiosarcoma; kidney cancers such as kidney adenocarcinoma, renal cell carcinoma, hypernephroma, and transitional cell carcinoma of the renal pelvis; bladder cancers; hematological cancers such as acute lymphocytic (lymphoblastic) leukemia, acute myeloid (myelocytic, myelogenous, myeloblastic, myelomonocytic) leukemia, chronic lymphocytic leukemia (e.g., Sezary syndrome and hairy cell leukemia), chronic myelocytic (myeloid, myelogenous, granulocytic) leukemia, Hodgkin's lymphoma, non-Hodgkin's lymphoma, B-cell lymphoma, mycosis fungoides, and myeloproliferative disorders (including myeloproliferative disorders such as polycythemia vera, myelofibrosis, thrombocythemia, and chronic myelocytic leukemia); skin cancers such as basal cell carcinoma, squamous cell carcinoma, melanoma, Kaposi's sarcoma, and Paget's disease; head and neck cancers; eye-related cancers such as retinoblastoma and intraoccular melanocarcinoma; male reproductive system cancers such as benign prostatic hyperplasia, prostate cancer, and testicular cancers (e.g., seminoma, teratoma, embryonal carcinoma, and choriocarcinoma); breast cancer; female reproductive system cancers such as uterine cancer (endometrial carcinoma), cervical cancer (cervical carcinoma), cancer of the ovaries (ovarian carcinoma), vulvar carcinoma, vaginal carcinoma, fallopian tube cancer, and hydatidiform mole; thyroid cancer (including papillary, follicular, anaplastic, or medullary cancer); pheochromocytomas (adrenal gland); noncancerous growths of the parathyroid glands; pancreatic cancers; and hematological cancers such as leukemias, myelomas, non-Hodgkin's lymphomas, and Hodgkin's lymphomas.

In some embodiments, the methods further comprise administering one or more chemotherapeutic agent. In some embodiments, the chemotherapeutic agent or agents are selected lymphodepleting (preconditioning) chemotherapeutic. Beneficial preconditioning treatment regimens are set forth in e.g., U.S. Pat. No. 9,855,298, along with correlative beneficial biomarkers described in PCT Patent Application PCT/US2016/034885, the contents of which are hereby incorporated by reference in their entirety herein. These describe, e.g., methods of conditioning a patient in need of a T-cell therapy comprising administering to the patient specified beneficial doses of cyclophosphamide (CYTOXAN™) (between about 200 mg/m²/day and about 2000 mg/m²/day) and specified doses of fludarabine (FLUDARA™) (between about 20 mg/m²/day and about 900 mg/m²/day). One such preferred dose regimen involves treating a patient comprising administering daily to the patient about 500-600 mg/m²/day of cyclophosphamide and about 30 mg/m²/day of fludarabine for three days prior to administration of a therapeutically effective amount of engineered T-cells to the patient. Preferred cell doses include, but are not limited to, 1×10⁶ to about 5×10⁶ engineered CART-cells/kg.

In some embodiments, the antigen binding molecule (such as a 4-1BB (CD137) agonist), transduced (or otherwise engineered) cells (such as CARs), and the chemotherapeutic agent are administered each in an amount effective to treat the disease or condition in the subject.

In some embodiments, compositions comprising CAR-expressing immune effector cells disclosed herein may be administered in conjunction with any number of chemotherapeutic agents. Examples of chemotherapeutic agents include alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine resume; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2, 2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel (TAXOL™, Bristol-Myers Squibb) and doxetaxel (TAXOTERE®, Rhone-Poulenc Rorer); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS2000; difluoromethylomithine (DMFO); retinoic acid derivatives such as Targretin™ (bexarotene), Panretin™, (alitretinoin); ONTAK™ (denileukin diftitox); esperamicins; capecitabine; and pharmaceutically acceptable salts, acids or derivatives of any of the above. In some embodiments, compositions comprising CAR- and/or TCR-expressing immune effector cells disclosed herein may be administered in conjunction with an anti-hormonal agent that acts to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above. Combinations of chemotherapeutic agents are also administered where appropriate, including, but not limited to CHOP, i.e., Cyclophosphamide (Cytoxan®), Doxorubicin (hydroxydoxorubicin), Vincristine (Oncovin®), and Prednisone.

In some embodiments, the chemotherapeutic agent is administered simultaneously, sequentially in any order or separately with the administration of a CD19-directed genetically modified T-cell immunotherapy and/or 4-1BB agonist. In some embodiments, the chemotherapeutic agent is administered simultaneously or within one week after the administration of a CD19-directed genetically modified T-cell immunotherapy and/or 4-1BB agonist. In other embodiments, the chemotherapeutic agent is administered from about 1 to about 4 weeks or from about 1 week to about 1 month, about 1 week to about 2 months, about 1 week to about 3 months, about 1 week to about 6 months, about 1 week to about 9 months, or about 1 week to about 12 months after the administration of a CD19-directed genetically modified T-cell immunotherapy and/or 4-1BB agonist. In some embodiments, the chemotherapeutic agent is administered at least 1 month before administering a CD19-directed genetically modified T-cell immunotherapy and/or 4-1BB agonist. In some embodiments, the methods further comprise administering two or more chemotherapeutic agents.

A variety of additional therapeutic agents may be used in conjunction with the compositions described herein. For example, potentially useful additional therapeutic agents include PD-1 inhibitors such as nivolumab (OPDIVO®), pembrolizumab (KEYTRUDA®), pembrolizumab, pidilizumab (CureTech), and atezolizumab (Roche).

Additional therapeutic agents suitable for use in combination with the compositions and methods disclosed herein include, but are not limited to, ibrutinib (IMBRUVICA®), ofatumumab (ARZERRA®), rituximab (RITUXAN®), bevacizumab (AVASTIN®), trastuzumab (HERCEPTIN®), trastuzumab emtansine (KADCYLA®), imatinib (GLEEVEC®), cetuximab (ERBITUX®), panitumumab (VECTIBIX®), catumaxomab, ibritumomab, ofatumumab, tositumomab, brentuximab, alemtuzumab, gemtuzumab, erlotinib, gefitinib, vandetanib, afatinib, lapatinib, neratinib, axitinib, masitinib, pazopanib, sunitinib, sorafenib, toceranib, lestaurtinib, axitinib, cediranib, lenvatinib, nintedanib, pazopanib, regorafenib, semaxanib, sorafenib, sunitinib, tivozanib, toceranib, vandetanib, entrectinib, cabozantinib, imatinib, dasatinib, nilotinib, ponatinib, radotinib, bosutinib, lestaurtinib, ruxolitinib, pacritinib, cobimetinib, selumetinib, trametinib, binimetinib, alectinib, ceritinib, crizotinib, aflibercept, adipotide, denileukin diftitox, mTOR inhibitors such as Everolimus and Temsirolimus, hedgehog inhibitors such as sonidegib and vismodegib, CDK inhibitors such as CDK inhibitor (palbociclib).

In some embodiments, the composition comprising CAR immune cells are administered with an anti-inflammatory agent. Anti-inflammatory agents or drugs can include, but are not limited to, steroids and glucocorticoids (including betamethasone, budesonide, dexamethasone, hydrocortisone acetate, hydrocortisone, hydrocortisone, methylprednisolone, prednisolone, prednisone, triamcinolone), nonsteroidal anti-inflammatory drugs (NSAIDS) including aspirin, ibuprofen, naproxen, methotrexate, sulfasalazine, leflunomide, anti-TNF medications, cyclophosphamide and mycophenolate. Exemplary NSAIDs include ibuprofen, naproxen, naproxen sodium, Cox-2 inhibitors, and sialylates. Exemplary analgesics include acetaminophen, oxycodone, tramadol of proporxyphene hydrochloride. Exemplary glucocorticoids include cortisone, dexamethasone, hydrocortisone, methylprednisolone, prednisolone, or prednisone. Exemplary biological response modifiers include molecules directed against cell surface markers (e.g., CD4, CD5, etc.), cytokine inhibitors, such as the TNF antagonists, (e.g., etanercept (ENBREL®), adalimumab (HUMIRA®) and infliximab (REMICADE®), chemokine inhibitors and adhesion molecule inhibitors. The biological response modifiers include monoclonal antibodies as well as recombinant forms of molecules. Exemplary DMARDs include azathioprine, cyclophosphamide, cyclosporine, methotrexate, penicillamine, leflunomide, sulfasalazine, hydroxychloroquine, Gold (oral (auranofin) and intramuscular), and minocycline.

In some embodiments, the compositions described herein are administered in conjunction with a cytokine. Examples of cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor (HGF); fibroblast growth factor (FGF); prolactin; placental lactogen; mullerian-inhibiting substance; mouse gonadotropin-associated peptide; inhibin; activin; vascular endothelial growth factor; integrin; thrombopoietin (TPO); nerve growth factors (NGFs) such as NGF-beta; platelet-growth factor; transforming growth factors (TGFs) such as TGF-alpha and TGF-beta; insulin-like growth factor-I and -II; erythropoietin (EPO, Epogen®, Procrit®); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines.

### Administration of Axicabtagene Ciloleucel and Utomilumab

Pharmacodynamics and Pharmacokinetics after Axi-cel™ Infusion

YESCARTA™ (axicabtagene ciloleucel; Axi-cel™; KTE-C19) is a CD19-directed genetically modified autologous chimeric antigen receptor (CAR) T-cell therapy that is approved by the United States Food and Drug Administration (FDA) for the treatment of adult patients with relapsed or refractory (r/r) large B-cell lymphoma after two or more lines of systemic therapy. Approved indications include diffuse large B-cell lymphoma (DLBCL) not otherwise specified, primary mediastinal large B-cell lymphoma (PMBCL), high grade B-cell lymphoma and DLBCL arising from follicular lymphoma.

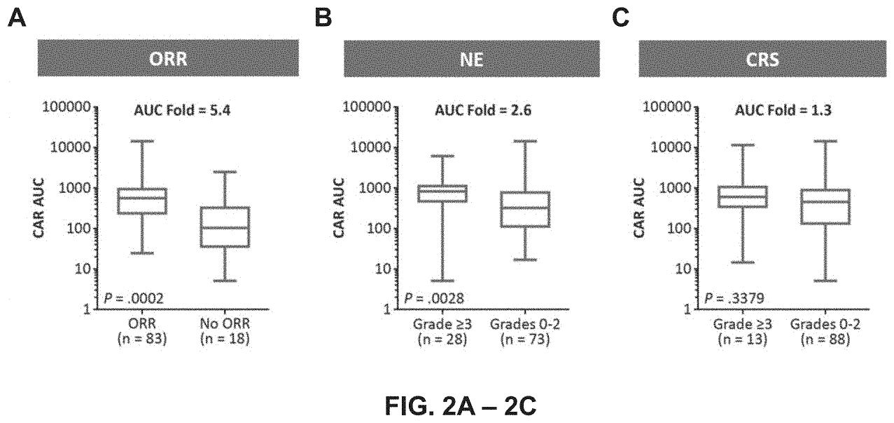

In the pivotal ZUMA-1 clinical trial of Axi-cel™ for the treatment of r/r large B-cell lymphoma key pharmacokinetic (PK) and pharmacodynamic (PD) relationships were elucidated that describe the relationship between anti-CD19 CAR T-cell expansion (PK) and levels of serum cytokines (PD) in relationship to clinical outcome (Locke et al. CT019—Primary results from ZUMA-1: a pivotal trial of axicabtagene ciloleucel (axicel; KTE-C19) in patients with refractory aggressive non-Hodgkin lymphoma (NHL). In: Proceedings of the 107th Annual Meeting of the American Association for Cancer Research; 2017 Apr. 1-5; Washington, D.C. Philadelphia (Pa.):AACR; 2017. Abstract 4292). FIG. 2 depicts the relationship between anti-CD19 CAR levels in blood over the first 28 days post infusion (AUC₀₋₂₈) with objective response (CR or PR), and development of Grade neurologic toxicity or cytokine release syndrome (CRS). Expansion of CAR T-cells was associated with objective response and grade neurologic toxicity but not grade CRS.

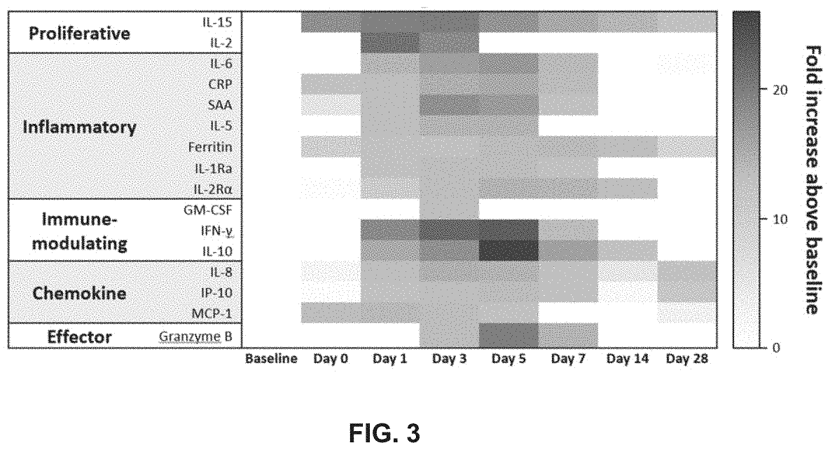

FIG. 3 highlights lymphodepletion chemotherapy and anti-CD19 CAR T induction of key immune programs over the first 28 days post infusion. Distinct biomarkers peak within 7 days after Axi-cel™ treatment. Analytes shown were evaluated in 50% of patients with 2-fold induction above baseline of a panel of 44 measured. Serum analytes were measured with MSD®, Luminex®, and Quantikine™ ELISA.

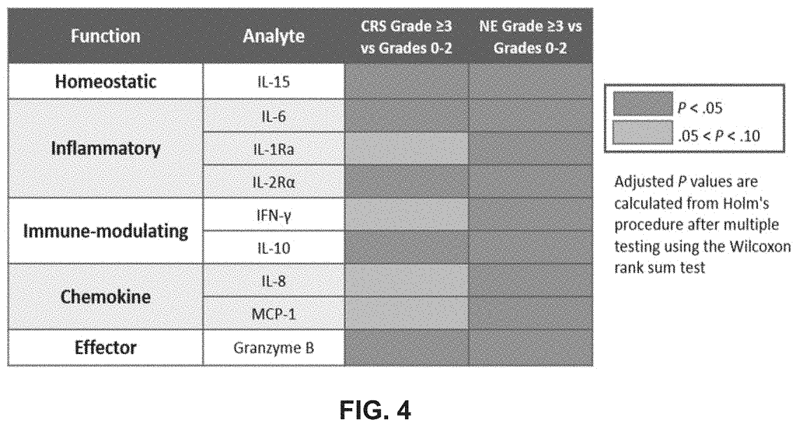

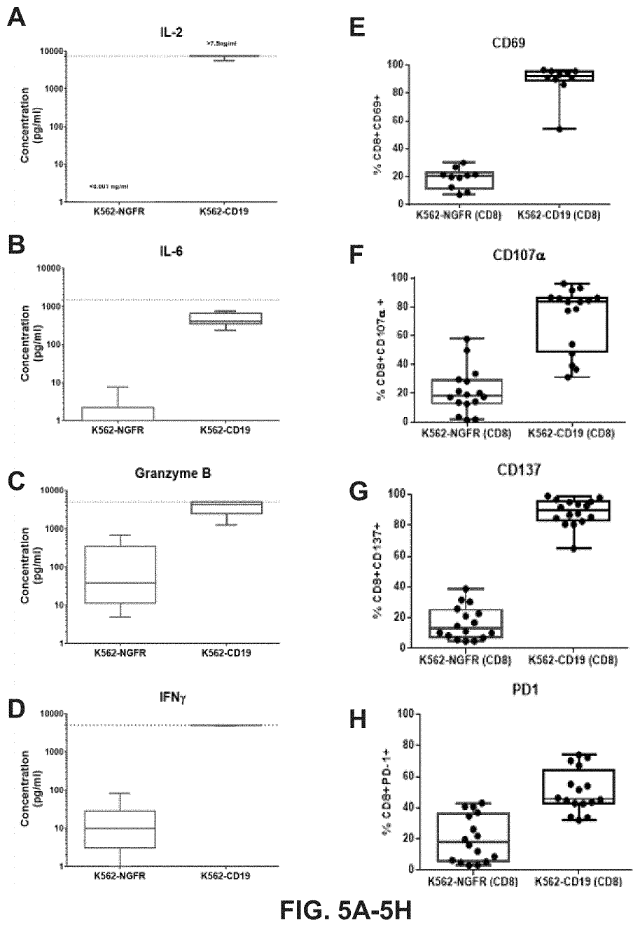

FIG. 4 shows biomarkers associated with both grade CRS and grade neurologic toxicity. The association between peak levels of serum analytes and association with Grade neurologic toxicity or CRS are shown. Peak levels after Axi-cel™ infusion were used in the comparison. Anti-CD19 CAR T-cells show broad polyfunctionality in co-culture (FIG. 5). The co-regulation of activation markers for T-cells gated on CD8+ cells across all products evaluated. The CD4+ T-cells demonstrated a similar pattern (data not shown). See Perez et al, ASH 2015, “Pharmacodynamic Profile and Clinical Response in Patients with B-Cell Malignancies of Anti-CD19 CART Cell Therapy” (Also as Abstract No. 2042).

Dosage and Administration of Axicabtagene Ciloleucel

In some embodiments, CD19-directed genetically modified autologous T-cell immunotherapy indicated for the treatment of adult patients with refractory large B-cell lymphoma is administered after two or more lines of systemic therapy. In some embodiments, an infusion bag of CD19-directed genetically modified autologous T-cell immunotherapy comprises a suspension of chimeric antigen receptor (CAR)-positive T-cells in approximately 68 mL. The target dose can be between about 1×10⁶ and about 2×10⁶ CAR-positive viable T-cells per kg body weight, with a maximum of 2×10⁸ CAR-positive viable T-cells. In some embodiments the CD19-directed genetically modified autologous T-cell immunotherapy is Axi-cel™ (YESCARTA™, axicabtagene ciloleucel).

In some embodiments, CD19-directed genetically modified autologous T-cell immunotherapy is for autologous use. The patient's identity must match the patient identifiers on the CD19-directed genetically modified autologous T-cell immunotherapy cassette and infusion bag. If the information on the patient-specific label does not match the intended patient, the CD19-directed genetically modified autologous T-cell immunotherapy cannot be administered.

In some embodiments, the availability of CD19-directed genetically modified autologous T-cell immunotherapy must be confirmed prior to starting the lymphodepleting regimen.

In some embodiments, the patient is pre-treated prior to CD19-directed genetically modified autologous T-cell immunotherapy infusion with administration of lymphodepleting chemotherapy. In some embodiments, a lymphodepleting chemotherapy regimen of cyclophosphamide about 500 mg/m² IV and fludarabine about 30 mg/m² IV on the fifth, fourth, and third day before infusion of CD19-directed genetically modified autologous T-cell immunotherapy is administered.

In some embodiments, the patient is premedicated prior to CD19-directed genetically modified autologous T-cell immunotherapy infusion by administration of acetaminophen at about 650 mg by mouth and diphenhydramine at about 12.5 mg intravenously or by mouth approximately 1 hour before CD19-directed genetically modified autologous T-cell immunotherapy infusion.

In some embodiments, the prophylactic use of systemic steroids is avoided as it may interfere with the activity of CD19-directed genetically modified autologous T-cell immunotherapy.

Preparation of CD19-Directed Genetically Modified Autologous T-Cell Immunotherapy for Infusion

The timing of CD19-directed genetically modified autologous T-cell immunotherapy thaw and infusion is coordinated. In some embodiments, the infusion time is confirmed in advance, and the start time of CD19-directed genetically modified autologous T-cell immunotherapy thaw is adjusted such that it will be available for infusion when the patient is ready.

In some embodiments, the patient identity is confirmed prior to CD19-directed genetically modified autologous T-cell immunotherapy thaw. Prior to CD19-directed genetically modified autologous T-cell immunotherapy preparation, patient's identity is matched with the patient identifiers on the CD19-directed genetically modified autologous T-cell immunotherapy cassette. In some embodiments, the CD19-directed genetically modified autologous T-cell immunotherapy product bag is not removed from the cassette if the information on the patient-specific label does not match the intended patient.

In some embodiments, once patient identification is confirmed, CD19-directed genetically modified autologous T-cell immunotherapy product bag is removed from the cassette and the patient information on the cassette label is confirmed to match the bag label.

In some embodiments, the method comprises inspecting the product bag for any breaches of container integrity such as breaks or cracks before thawing. In some embodiments, the infusion bag is placed inside a second sterile bag per local guidelines.

In some embodiments, the method comprises thawing the CD19-directed genetically modified autologous T-cell immunotherapy at approximately 37° C. using either a water bath or dry thaw method until there is no visible ice in the infusion bag. In some embodiments, the method comprises mixing or agitating the contents of the bag to disperse clumps of cellular material. In some embodiments, the contents of the bag are gently mixed or agitated. In some embodiments, the method comprises inspecting the bag for the presence of visible cell clumps remaining and mixing or agitation is continued. Small clumps of cellular material should disperse with gentle manual mixing. In some embodiments, the method does not comprise a wash, spin down, and/or re-suspension of CD19-directed genetically modified autologous T-cell immunotherapy in new media prior to infusion.

In some embodiments, once thawed, CD19-directed genetically modified autologous T-cell immunotherapy may be stored at room temperature (20° C. to 25° C.) for up to 3 hours.

### Administration

In some embodiments, the presently disclosed methods of administration of CD19-directed genetically modified autologous T-cell immunotherapy comprise one or more of the following as steps or as considerations:
Ensure that tocilizumab and emergency equipment are available prior to infusion and during the recovery period.
Do NOT use a leukodepleting filter.
Central venous access is recommended for the infusion of CD19-directed genetically modified autologous T-cell immunotherapy.
Confirm the patient's identity matches the patient identifiers on the CD19-directed genetically modified autologous T-cell immunotherapy product bag.
Prime the tubing with normal saline prior to infusion.
Infuse the entire contents of the CD19-directed genetically modified autologous T-cell immunotherapy bag within 30 minutes by either gravity or a peristaltic pump. CD19-directed genetically modified autologous T-cell immunotherapy is stable at room temperature for up to 3 hours after thaw.
Gently agitate the product bag during CD19-directed genetically modified autologous T-cell immunotherapy infusion to prevent cell clumping.
After the entire content of the product bag is infused, rinse the tubing with normal saline at the same infusion rate to ensure all product is delivered.
CD19-directed genetically modified autologous T-cell immunotherapy contains human blood cells that are genetically modified with replication incompetent retroviral vector. Follow universal precautions and local biosafety guidelines for handling and disposal to avoid potential transmission of infectious diseases.

### Monitoring

In some embodiments, administration of CD19-directed genetically modified autologous T-cell immunotherapy occurs at a certified healthcare facility. In some embodiments, the methods disclosed herein comprise monitoring patients at least daily for 7 days at the certified healthcare facility following infusion for signs and symptoms of CRS and neurologic toxicities. In some embodiments, patients are instructed to remain within proximity of the certified healthcare facility for at least 4 weeks following infusion.

Management of Severe Adverse Reactions

In some embodiments, the method comprises management of adverse reactions. In some embodiments, the adverse reaction is selected from the group consisting of cytokine release syndrome (CRS), a neurologic toxicity, a hypersensitivity reaction, a serious infection, a cytopenia and hypogammaglobulinemia.

In some embodiments, the signs and symptoms of adverse reactions are selected from the group consisting of fever, hypotension, tachycardia, hypoxia, and chills, include cardiac arrhythmias (including atrial fibrillation and ventricular tachycardia), cardiac arrest, cardiac failure, renal insufficiency, capillary leak syndrome, hypotension, hypoxia, organ toxicity, hemophagocytic lymphohistiocytosis/macrophage activation syndrome (HLH/MAS), seizure, encephalopathy, headache, tremor, dizziness, aphasia, delirium, insomnia anxiety, anaphylaxis, febrile neutropenia, thrombocytopenia, neutropenia, and anemia.

### 4-1BB (CD-137) Protein Receptor Agonists

The 4-1BB (CD-137) protein receptor is found on certain T-cells (primarily on CD8+, but also on CD4+ memory T-cells) and natural killer (NK) cells. (Fisher, T. S., Kamperschroer, C., Oliphant, T. et al. Cancer Immunol Immunother (2012) 61: 1721. https://doi.org/10.1007/s00262-012-1237-1; Westwood J A, Potdevin Hunnam T C U, Pegram H J, Hicks R J, Darcy P K, Kershaw M H (2014) Routes of Delivery for CpG and Anti-CD137 for the Treatment of Orthotopic Kidney Tumors in Mice. PLoS ONE 9(5): e95847. https://doi.org/10.1371/journal.pone.0095847). 4-1BB may also be referred to as TNFRSF9; tumor necrosis factor receptor superfamily, member 9; ILA; 4-1BB; CD137; CDw137; tumor necrosis factor receptor superfamily member 9; CD137 antigen. 4-1BB binding molecules, including utomilumab are further described in U.S. Pat. No. 8,337,850, which is hereby incorporated by reference in its entirety.

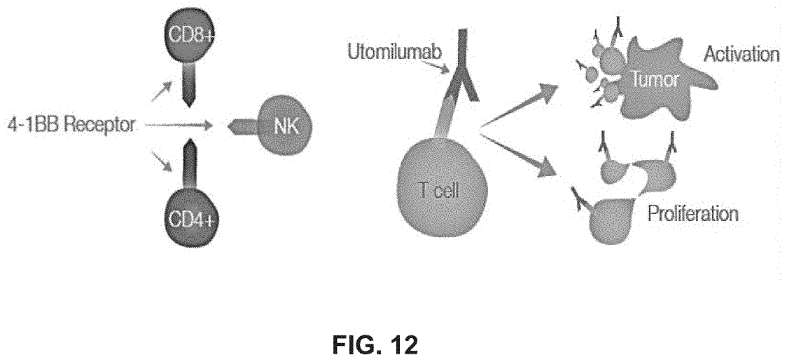

Utomilumab is the non-proprietary name for PF-05082566, an investigational immunotherapy and fully human IgG2 monoclonal antibody (mAb). As shown in FIG. 12, when utomilumab (PF-05082566) binds to 4-1BB, it has been observed to stimulate and increase the number of immune cells. Combining utomilumab (PF-05082566) with a checkpoint inhibitor, such as anti-PD-1/anti-PD-L1, or other immunotherapies may amplify the immune response. (Gopal A, Barlett N, Levy R, et al. A Phase I study of PF-05082566 (anti-4-1BB)+rituximab in patients with CD20+ NHL. J Clin Oncol 33, 2015 DOI: 10.1200/jco.2015.33.15_supp1.3004; Tolcher M D, Anthony W. Phase 1b trial investigating utomilumab (a 4-1BB agonist) in combination with a checkpoint inhibitor. Oral presentation at the 52nd Annual Meeting of the American Society of Clinical Oncology 2016 (ASCO). http://meetinglibrary.asco.org/content/125783?media=sl. Accessed Feb. 27, 2017.; A Study of PF-05082566 as a Single Agent and in Combination with Rituximab. https://clinicaltrials.gov/ct2/show/NCT01307267?term=PF-05082566&rank=3. Accessed Feb. 27, 2017.)

In some embodiments, the 4-1BB (CD137) agonist is an isolated antibody, or antigen-binding portion thereof, comprising three CDRs of a VH region amino acid sequence as set forth in SEQ ID NO:1 and three CDRS of a VL region amino acid sequence set forth in SEQ ID NO: 3.

In some embodiments, the 4-1BB agonist is an isolated antibody, or antigen-binding portion thereof, comprising: (a) a H-CDR1 as set forth in SEQ ID NO:5; (b) a H-CDR2 as set forth in SEQ ID NO:6; (c) a H-CDR3 as set forth in SEQ ID NO:7; (d) a L-CDR1 as set forth in SEQ ID NO:8; (e) a L-CDR2 as set forth in SEQ ID NO:9; and (f) a L-CDR3 as set forth in SEQ ID NO:10.

In some embodiments, the 4-1BB agonist is an isolated antibody, or antigen-binding portion thereof, comprising a VH region amino acid sequence as set forth in SEQ ID NO:1. In some embodiments, the antibody, or antigen-binding portion comprises a VL region amino acid sequence as set forth in SEQ ID NO:3.

In some embodiments, the 4-1BB agonist is an isolated antibody, or antigen-binding portion thereof, comprising a VH region amino acid sequence as set forth in SEQ ID NO:1 and a VL region amino acid sequence as set forth in SEQ ID NO:3.

In some embodiments, the 4-1BB agonist isolated antibody is an IgG2. In some embodiments, the 4-1BB agonist is a fully human antibody.

In some aspects, the present invention provides a pharmaceutical composition comprising an antibody, or antigen-binding portion thereof, described herein and a pharmaceutically acceptable carrier.

In some embodiments, the 4-1BB agonist isolated antibody comprises a heavy chain amino acid sequence as set forth in SEQ ID NO:2 and a light chain amino acid sequence as set forth in SEQ ID NO:4, with the proviso that the C-terminal lysine residue of SEQ ID NO:2 is optionally absent.

Dosage and Administration of Utomilumab

The term “therapeutically effective amount” or “therapeutically effective dose” of a binding molecule refers to an amount that is effective for an intended therapeutic purpose. For example, in the treatment of cancer, examples of desirable or beneficial effects include inhibition of further growth or spread of cancer cells, death of cancer cells, inhibition of reoccurrence of cancer, reduction of pain associated with the cancer, or improved survival of the mammal. The therapeutically effective amount of a 4-1BB antibody usually ranges from about 0.001 to about 500 mg/kg, and more usually about 0.01 to about 200 mg/kg, of the body weight of the mammal. For example, the amount can be about 0.3 mg/kg, about 1 mg/kg, about 3 mg/kg, about 5 mg/kg, about 10 mg/kg, about 50 mg/kg, about 100 mg/kg, or about 200 mg/kg of body weight of the mammal. In some embodiments, the therapeutically effective amount of a 4-1BB antibody is in the range of about 0.01-30 mg/kg of body weight of the mammal. In some other embodiments, the therapeutically effective amount of a 4-1BB antibody is in the range of about 0.05-15 mg/kg of body weight of the mammal. The precise dosage level to be administered can be readily determined by a person skilled in the art and will depend on a number of factors, such as the type, and severity of the disorder to be treated, the particular binding molecule employed, the route of administration, the time of administration, the duration of the treatment, the particular additional therapy employed, the age, sex, weight, condition, general health and prior medical history of the patient being treated, and like factors well known in the medical arts.

In some embodiments, the therapeutically effective dose of a 4-1BB antibody is between about 1 to about 200 mg. In some embodiments, the therapeutically effective dose of a 4-1BB antibody is between about 1 to about 100 mg. A binding molecule or composition is usually administered on multiple occasions. Intervals between single doses can be, for example, weekly, monthly, every three months or yearly. An exemplary treatment regimen entails administration once per week, once every two weeks, once every three weeks, once every four weeks, once a month, once every three months or once every three to six months. Dosage regimens for a 4-1BB antibody can include about 1 mg/kg body weight or about 3 mg/kg body weight via intravenous administration, using one of the following dosing schedules: (i) every four weeks for six dosages, then every three months; (ii) every three weeks; (iii) about 3 mg/kg body weight once followed by about 1 mg/kg body weight every three weeks.

In some embodiments, a 4-1BB agonist fully human monoclonal antibody is administered at a dose of about 1 mg, about 2 mg, about 3 mg, about 4 mg, about 5 mg, about 7.5 mg, about 10 mg, about 15 mg, about 20 mg, about 25 mg, about 30 mg, about 40 mg, about 50 mg, about 75 mg, about 100 mg, about 125 mg, about 150 mg, about 175 mg, about 200 mg. In some embodiments, a 4-1BB agonist fully human monoclonal antibody dosing continues until patients demonstrate complete remission, non-response/progressive disease, or for about 1 year. In some embodiments, a 4-1BB agonist fully human monoclonal antibody is administered about every 4 weeks. In some embodiments, a 4-1BB agonist fully human monoclonal antibody is administered monthly.

The present disclosure is further illustrated by the following examples which should not be construed as further limiting. The contents of all figures and all references, patents and published patent applications cited throughout this disclosure are expressly incorporated herein by reference in their entirety.

### EXAMPLES

### Example 1: Clinical Studies of Refractory Large B-Cell Lymphoma

Combination therapy with axicabtagene ciloleucel in combination with utomilumab can be used to effectively treat cancer patients. This example illustrates a multi-center study evaluating the safety and efficacy of KTE-C19 (axicabtagene ciloleucel) in combination with utomilumab in subjects with Refractory Large B-cell Lymphoma or Refractory Diffuse Large B-Cell Lymphoma (DLBCL) after at least 2 prior lines of systemic therapy. The trial is separated into two distinct phases designated as Phase 1 and Phase 2.

During Phase 1 approximately 3-9 or 3-24 subjects with refractory large B-cell lymphoma or refractory DLBCL are enrolled in a 3+3 design in up to 3 or 4 of 6 cohorts to evaluate the safety of KTE-C19 and utomilumab combination regimens. KTE-C19 is administered at a fixed or single dose, and the utomilumab dose administered at escalating doses or is increased sequentially in each of the 3 cohorts. The primary objective of Phase 1 is to evaluate the safety of the KTE-C19 and utomilumab combination regimens, and to identify the most appropriate dose and timing of utomilumab to carry forward into the Phase 2. Incidence of adverse events defined as dose-limiting toxicities (DLT) is a primary endpoint.

In Phase 2, approximately 22 or 24 subjects are enrolled to receive combination treatment with KTE-C19 and utomilumab based on the dose and schedule selected following a review of the data from the Phase 1 portion. The primary objective of Phase 2 is to evaluate the efficacy of KTE-C19 and utomilumab, as measured by the complete response (CR) rate in subjects with refractory large B-cell lymphoma or refractory DLBCL. Secondary objectives include an assessment of the safety and tolerability of KTE-C19 in combination with utomilumab and the evaluation of additional efficacy endpoints. Primary endpoint of Phase 2 is complete response rate (complete response [CR] per the revised International Working Group [IWG]) Response Criteria for Malignant Lymphoma (Cheson et al. J Clin Oncol 25:579-586 (2007)) or Lugano Classification (Cheson et al., 2014), as determined by study investigators.

Independent of the cohort or phase of the study, each subject proceeds through the following study periods:
Screening
Enrollment/Leukapheresis
Bridging therapy, if applicable
Conditioning chemotherapy
Combination treatment (KTE-C19 and utomilumab)
Post-treatment assessment
Long term follow-up

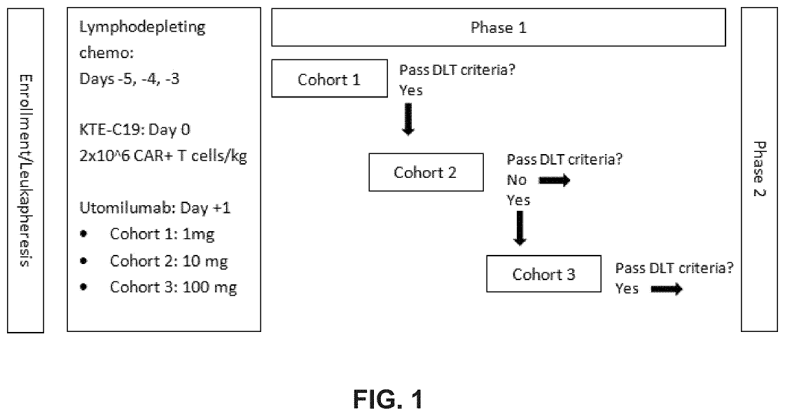

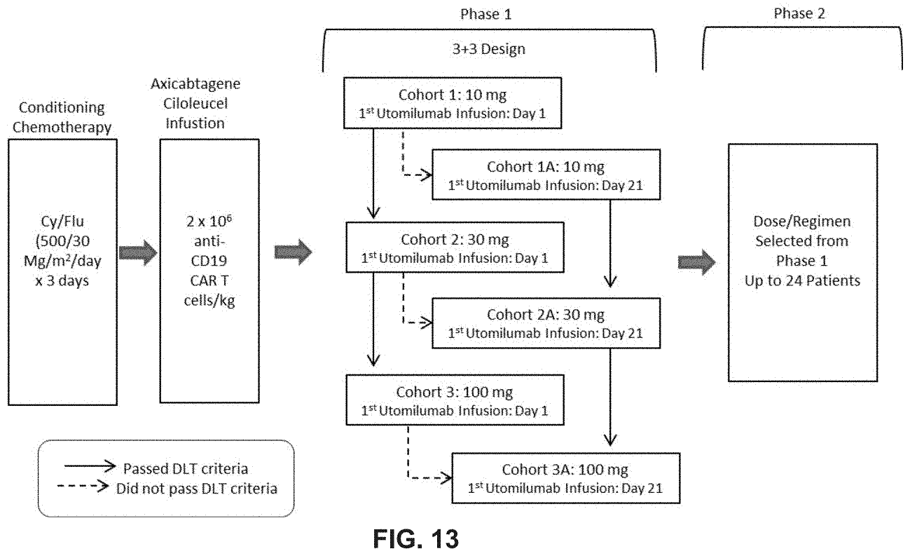

As shown in FIG. 1 and FIG. 13, patients first undergo enrollment and leukapheresis, followed by conditioning lymphodepleting chemotherapy on days −5, −4, and −3 prior to the start of combination treatment with KTE-C19 and utomilumab. Patients receive a conditioning chemotherapy regimen consisting of fludarabine 30 mg/m²/day and cyclophosphamide 500 mg/m²/day, administered for 3 days. On day 0, KTE-C19 treatment comprises a single infusion of CAR transduced autologous T-cells administered intravenously at a target dose of 2×10⁶ anti-CD19 CAR T-cells/kg. Under circumstances where subjects initially respond and subsequently relapse, subjects may be eligible for a second course of conditioning chemotherapy and KTE-C19.

Utomilumab treatment comprises an intravenous infusion given about every four weeks. The first dose is administered the day following KTE-C19 infusion, and dosing continues until patients demonstrate complete remission, non-response/progressive disease, or for about 1 year, whichever is sooner. In one study design shown in FIG. 1, Cohort 1 subjects receive about 1 mg of utomilumab, Cohort 2 subjects receive about 10 mg of utomilumab, and Cohort 3 subjects receive about 100 mg of utomilumab. In another study design shown in FIG. 13, utomilumab will begin at a fixed dose of 10 mg on Day 1 in Cohort 1 and the utomilumab regimens administered are outlined in Table 1 below.

TABLE 1
Utomilumab Regimens

| | | First Utomilumab |
|---|---|---|
| Dose Level | Cohort | Administration |
| 10 mg | 1 | Day 1 |
| | 1A | Day 21 |
| 30 mg | 2 | Day 1 |
| | 2A | Day 21 |
| 100 mg | 3 | Day 1 |
| | 3A | Day 21 |

At specific time points, subjects undergo the following procedures: collection of informed consent, general medical history including previous treatments for NHL, physical exam including vital signs and performance status, and neurological assessments. Subjects also undergo blood draws for complete blood count (CBC), chemistry panels, cytokines, C-reactive protein, lymphocyte subsets, anti-KTE-C19 antibodies, ADA assessment, replication competent retrovirus (RCR) and anti-CD19 CAR T-cell analysis. Women of child-bearing potential undergo a urine or serum pregnancy test.

Subjects also undergo a baseline electrocardiogram (ECG), echocardiogram (ECHO), brain magnetic resonance image (MRI), a positron emission tomography-computed tomography (PET-CT), and leukapheresis.

Objective Response Rate (CR+PR) is be determined per the revised IWG Response Criteria for Malignant Lymphoma (Cheson, 2007) and determined by IWG Response Criteria for Malignant Lymphoma or Lugano Classification (Cheson et al. Journal of Clinical Oncology 32, no. 27 (September 2014) 3059-3067). Duration of Response is assessed. The Progression-Free Survival (PFS) by investigator assessment per Lugano Response Classification Criteria (Cheson et al., 2014) is evaluated.

Pharmacokinetics determinations include PK parameters of utomilumab as data permit: maximum plasma concentration (Cmax), time to maximum plasma concentration (Tmax), area under the plasma concentration time curve from time 0 to τ hours post dose (AUC_{0-τ}, where τ is dependent on the analyte) apparent plasma clearance (CL/F) or systemic clearance (CL), and apparent volume of distribution (V/F) or steady state volume of distribution (Vss) of each analyte following single and multiple dosing.

Molecular, cellular, and soluble markers in peripheral blood and/or tumor tissue and/or feces that may be relevant to the mechanism of action of, or response/resistance to study treatment, including molecular profiling for ABC/GBC cell of origin DLBCL subtypes are evaluated.

Overall survival and incidence of adverse events and clinically significant changes in safety lab values are determined. Further, incidence of anti-KTE-C19 antibodies and immunogenicity evaluations of anti-drug antibodies (ADA) and neutralizing antibodies (Nab) against utomilumab is assessed.

PD-L1 expression levels in tumor cells and cells of the tumor microenvironment at baseline, levels of KTE-C19 in blood, and levels of cytokines and other markers in serum can are also assessed during the study.

In addition, exploratory studies are performed to explore at baseline and post-treatment molecular, cellular, and soluble markers (for example, but not limited to, baseline mutational profile, baseline microbiome profile, baseline and changes in gene expression profiles, tumor infiltrating lymphocytes and cytokine levels) in peripheral blood and/or tumor tissue and/or feces that may be relevant to the mechanism of action of, or response/resistance to, study treatment.

Frequencies of utomilumab dose delays for ongoing acute toxicities following KTE-C19 are also evaluated. This study utilizes a single-arm design to estimate the true complete response rate in patients with refractory large B-cell lymphoma or relapsed or refractory DLBCL treated with the combination of utomilumab and KTE-C19. With a total sample size of 25 or 27 patients at any given dosing schedule, of which at least 3 patients have been treated in the Phase 1 portion, an observed CR rate of 60% yields 95% confidence that the estimate of the true CR rate is between 39% and 79%, or the maximum half-width of 95% confidence interval that estimate the true CR rate is no greater than 21%.

Study eligibility criteria are outlined below:

In one aspect, Key Inclusion Criteria include:
Histologically proven large B-cell lymphoma including the following types defined by (Swerdlow et al, 2016):
   DLBCL not otherwise specified (ABC/GCB)
   HGBCL with or without MYC and BCL2 and/or BCL6 rearrangement
   DLBCL arising from FL
   T cell/histiocyte rich large B-cell lymphoma
   DLBCL associated with chronic inflammation
   Primary cutaneous DLBCL, leg type
   Epstein-Barr virus (EBV)+DLBCL
Chemotherapy-refractory disease, defined as one or more of the following:
   No response to second or greater lines of therapy
      PD as best response to most recent therapy regimen
      SD as best response after at least 2 cycles of last line of therapy with SD duration no longer than 6 months from last dose of therapy

OR
Refractory post-ASCT
   Disease progression or relapsed ≤12 months after ASCT (must have biopsy proven recurrence in relapsed subjects)
   if salvage therapy is given post-ASCT, the subject must have had no response to or relapsed after the last line of therapy
At least 1 measurable lesion according to the Lugano Classification (Cheson et al. 2014). Lesions that have been previously irradiated will be considered measurable only if progression has been documented following completion of radiation therapy
Subject must have received adequate prior therapy including at minimum:
   Anti-CD20 monoclonal antibody unless investigator determines that tumor is CD20-negative, and
   An anthracycline containing chemotherapy regimen
No evidence, suspicion and/or history of central nervous (CNS) involvement of lymphoma
At least 2 weeks or 5 half-lives, whichever is shorter, must have elapsed since any prior systemic therapy at the time the subject is planned for leukapheresis
Toxicities due to prior therapy must be stable and recovered to Grade 1 (except for clinically non-significant toxicities such as alopecia)
   Age 18 or older
   Eastern Cooperative Oncology Group (ECOG) performance status of 0 or 1
   Absolute neutrophil count (ANC)≥1000/μL
   Platelet count ≥75,000/4;
   Absolute lymphocyte count ≥100/μL
   Adequate renal, hepatic, pulmonary, and cardiac function defined as:
Creatinine clearance (as estimated by Crockcroft Gault)≥60 mL/min
Serum alanine aminotransferase/aspartate aminotransferase ALT/AST ≤2.5 upper limit of normal (ULN)
Total bilirubin ≤1.5 mg/dL, except in subjects with Gilbert's syndrome.
Cardiac ejection fraction ≥50% and no evidence of pericardial effusion within 180 days provided the subject did not receive an anthracycline-based treatment or experience a cardiac event or change in performance status
No clinically significant pleural effusion
Baseline oxygen saturation >92% on room air
Females of childbearing potential must have a negative serum or urine pregnancy test (females who have undergone surgical sterilization or who have been postmenopausal for at least 2 years are not considered to be of childbearing potential)
   In another aspect, Key Inclusion Criteria include:
Histologically confirmed DLBCL
Documentation that the disease is refractory following at least 2 lines of systemic therapy
Documentation of baseline measurable disease
A biopsy (archived or Screening/recent) is be collected at Screening
Estimated life expectancy months
At least 18 years of age
Eastern Cooperative Oncology Group (ECOG) Performance Status (PS) 0 or 1
Patients must have an adequate bone marrow function, including:
Absolute neutrophil count (ANC)≥1.5×10⁹/L;
Platelet count ≥100×10⁹/L;
Hemoglobin ≥8 g/dL.
Patients must have adequate liver function, including:
Total bilirubin level ≤1.5×upper limit of normal (ULN);
Aspartate aminotransferase (AST) and alanine aminotransferase (ALT)≤2.5×ULN.
Patients must have an adequate renal function as evidenced by a creatinine clearance ≥60 mL/min as calculated using the Cockcroft-Gault equation.
   In one aspect, Key Exclusion Criteria include:
Histologically proven PMBCL
History of Richter's transformation of CLL
Prior non-drug anti-cancer therapy including chimeric antigen receptor (CAR) T-cell (CAR T-Cell) therapy or other genetically modified T-cell therapy
History of severe, immediate hypersensitivity reaction attributed to aminoglycosides
Presence or suspicion of fungal, bacterial, viral, or other infection that is uncontrolled or requiring IV antimicrobials for management. Simple UTI and uncomplicated bacterial pharyngitis are permitted if responding to active treatment and after consultation with the sponsor's Medical Monitor
History of HIV infection or acute or chronic active hepatitis B or C infection. Subjects with history of hepatitis infection must have cleared their infection as determined by standard serological and genetic testing per current Infectious Diseases Society of America (IDSA) guidelines or applicable country guidelines
Presence of any indwelling line or drain (eg, percutaneous nephrostomy tube, indwelling Foley catheter, biliary drain, or pleural/peritoneal/pericardial catheter). Dedicated central venous access catheters, such as a Port-a-Cath or Hickman catheter, are permitted
Subjects with detectable cerebrospinal fluid malignant cells, brain metastases, or a history of central nervous system (CNS) lymphoma based on clinical evaluation
History or presence of CNS disorder, such as seizure disorder, cerebrovascular ischemia/hemorrhage, dementia, cerebellar disease, or any autoimmune disease with CNS involvement
Subjects with cardiac atrial or cardiac ventricular lymphoma involvement
History of myocardial infarction, cardiac angioplasty or stenting, unstable angina, or other clinically significant cardiac disease within 12 months of enrollment
Requirement for urgent therapy due to tumor mass effects (eg, blood vessel compression, bowel obstruction, or transmural gastric involvement)
Primary immunodeficiency
History of autoimmune disease (eg, Crohn's, rheumatoid arthritis, systemic lupus) resulting in end organ injury or requiring systemic immunosuppression/systemic disease modifying agents within the last 2 years. Patients with a history of autoimmune-related hypothyroidism on a stable dose of thyroid replacement hormone and patients with controlled type 1 diabetes mellitus on a stable insulin regimen may be eligible for this study
History of symptomatic deep vein thrombosis or pulmonary embolism within 6 months of enrollment
Any medical condition likely to interfere with assessment of safety or efficacy of study treatment
History of severe immediate hypersensitivity reaction to any of the agents used in this study
Live vaccine ≤6 weeks prior to planned start of conditioning chemotherapy
Women of childbearing potential who are pregnant or breastfeeding because of the potentially dangerous effects of the preparative chemotherapy on the fetus or infant. Females who have undergone surgical sterilization or who have been postmenopausal for at least 2 years are not considered to be of childbearing potential.
Subjects of both genders who are not willing to practice birth control from the time of consent through 90 days after the last dose of utomilumab and at least 6 months after the completion of conditioning chemotherapy
History of malignancy other than nonmelanoma skin cancer in situ (eg, cervix, bladder, breast) or low-grade (Gleason ≤6) prostate cancer or surveillance without any plans for treatment, unless disease free for a least 3 years
Autologous stem cell transplant within 6 weeks of planned enrollment
Prior organ transplantation including prior allogeneic stem cell transplantation (SCT)
Prior CD19 targeted therapy with the exception of subjects who received axicabtagene ciloleucel (KTE-C19) in this study and are eligible for re-treatment
Use of any standard or experimental anti-cancer therapy within 2 weeks prior to enrollment, including cytoreductive therapy and radiotherapy, immunotherapy, or cytokine therapy (except for erythropoietin)
Prior treatment with PD-L1 inhibitor, PD-1 inhibitor, anti-CTLA4, anti-CD137 (4-1BB), anti-OX40 or other immune checkpoint blockade or activator therapy
Treatment with systemic immunostimulatory agents (including but not limited to interferon and IL-2) within 6 weeks or 5 half-lives of the drug, whichever is shorter, prior to the first utomilumab dose
History of idiopathic pulmonary fibrosis, organizing pneumonia (eg, bronchiolitis obliterans), drug-induced pneumonitis, idiopathic pneumonitis, or evidence of active pneumonitis per chest CT scan at screening. History of radiation pneumonitis in the radiation field (fibrosis) is allowed
In the investigator's judgment, the subject is unlikely to complete all protocol-required study visits or procedures, including follow-up visits, or comply with the study requirements for participation.
   In another aspect, Key Exclusion Criteria include:
Symptomatic central nervous system (CNS) lymphoma based on clinical evaluation
Prior organ transplantation including prior allogeneic SCT
Prior therapy with a 4-1BB agonist
Use of any standard or experimental anti-cancer therapy within 2 weeks prior to enrollment, including cytoreductive therapy and radiotherapy, immunotherapy, or cytokine therapy (except for erythropoietin)
Autologous stem cell transplant within 3 weeks of enrollment
Prior CD19 targeted therapy with the exception of subjects who received KTE-C19 in this study and are eligible for re-treatment
Use of any non-drug anti-cancer therapy including chimeric antigen receptor (CAR) T-Cell (CART-Cell) therapy.
History of autoimmune disease, requiring systemic immunosuppression within the last 2 years.
Diagnosis of any other malignancy years prior to first dose of study treatment, with the exception of: (i) adequately treated basal cell or squamous cell skin cancer, (ii) carcinoma in situ of the breast or cervix, or (iii) low-grade (Gleason 6) prostate cancer on surveillance without any plans for treatment intervention (e.g., surgery, radiation, or castration).

### Example 2: Assessment Plan for Axicabtagene Ciloleucel (KTE-C19, Axi-cel™) and Utomilumab

Certain aspects of resistance to KTE-C19 will be further assessed. The treatment assessment plan, comprising evaluation of pharmacokinetics, pharmacodynamics, tumor and immune biomarkers and also product characteristics, supports the multi-center study evaluating the safety, efficacy and mechanism of action of Axi-cel™ in combination with the 4-1BB (CD137) agonist antibody utomilumab in subjects with refractory large B-cell lymphoma or refractory DLBCL described in Example 1. Through translational analysis, the assessment plan determines if rapid upregulation of anti-CD19 CAR T-cell surface CD137 levels leads to responsiveness to agonist driven activation, leading to increased expansion and clinical activity. The mechanism of action of resistance in the tumor microenvironment (TME) and mechanisms of neurological toxicity (CSF) can also be investigated.

Analysis is performed of paired (pre and post-dose) core needle tumor biopsies harvested at time points coinciding with peak peripheral anti-CD19 CAR T-cell expansion to better understand the biology of CAR T-cells in the TME and the possible impact of utomilumab. Core needle biopsy is performed with a computed tomography (CT) or ultrasound guided core needle biopsy procedure using either an 18G or 20G needle is performed according to institutional guidelines to obtain 3-6 tumor core samples. An immunohistochemistry (IHC) and RNA transcript profiling analysis is performed on formalin fixed paraffin embedded (FFPE) or frozen tumor tissue from pre and post-dose core needle biopsies from patients with refractory large B-cell lymphoma or r/r DLBCL. Flow cytometric methods are also utilized for analysis of cryopreserved BM.

The assessment plan can include an aggressive collection strategy to gather cerebrospinal fluid (CSF) in subjects that are observed to develop Grade 2 or higher neurologic toxicity to understand mechanism of action of resistance in the TME and also mechanisms of neurological toxicity (CSF).

Sample collection and analysis strategies may provide direct evidence of CAR T-cell migration into the tumor microenvironment as well as activation, on-target cellular destruction and persistence. Evaluation of tumor cell characteristics and microenvironment can establish CAR efficacy in relation to molecular and histological disease features.

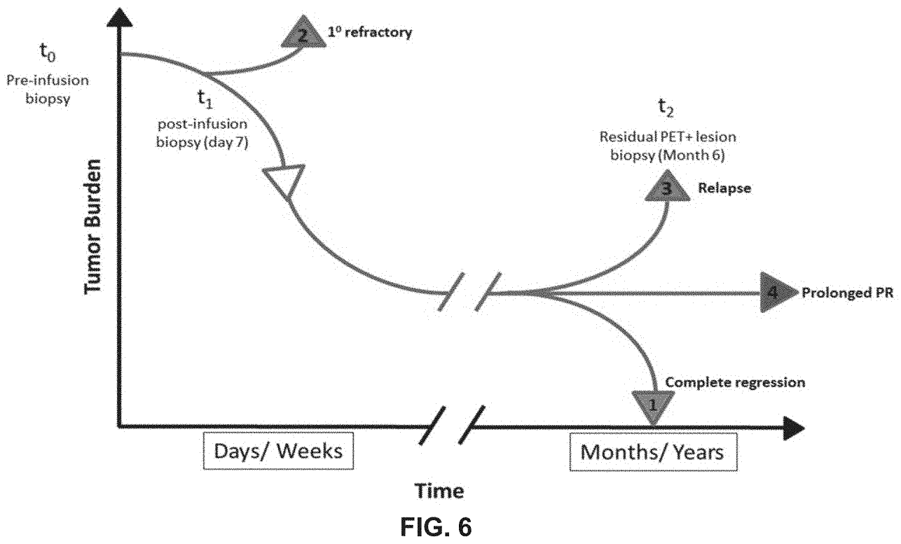

The biomarker collection strategy (FIG. 6) builds a sample bank derived from treated patients that fall into four broad categories of response as defined by objective response features: 1) regression [complete response (CR) or partial response (PR)], 2) refractory to treatment [progressive disease (PD)], 3) relapse or 4) persisting without evidence of progression or complete regression [prolonged PR or stable disease (SD)]. Evaluation of persisting disease (prolonged PR or SD) provides mechanistic insight with regards to immune confinement of tumor lesions. In addition, data derived from paired biopsy material can elucidate potential mechanisms of resistance or relapse that enables both rational design of next generation CAR products and clinical trial designed to utilize combinatorial approaches geared towards boosting immune response.

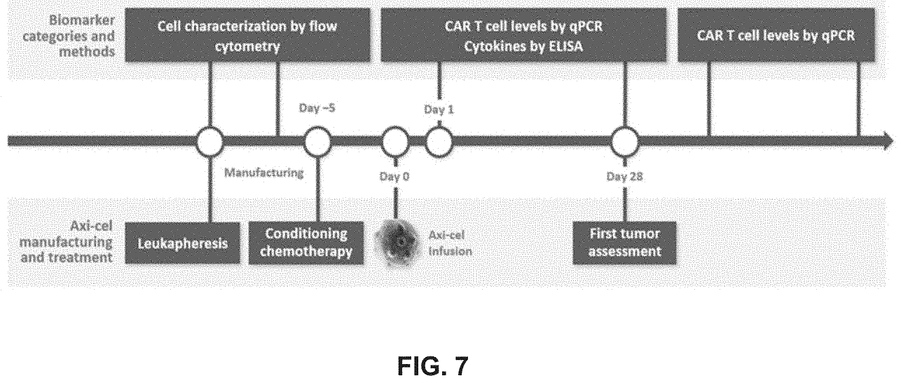

A generalized collection schedule for archival tumor, blood [peripheral blood mononuclear cells (PBMC), serum/plasma] and CSF samples intended for analysis are summarized in FIG. 7. The blood collection strategy includes sample draws at baseline, days 7, 14, 28 and months 3, 6, 9, 12, 15, 18, 24 and also at Days 2 and 6 after each utomilumab administration. Blood samples are used for determination of anti-CD19 CAR T-cell and serum biomarker (cytokine) levels.

Flow cytometry assays are performed for evaluation of leukocyte subsets present prior to transduction/expansion and also T-cell activation status in patient apheresis material. Cyropreserved patient apheresis material is assessed with Apheresis Panel 1 and 2 summarized in Table 2.

TABLE 2
Flow cytometry panel for apheresis characterization

| Apheresis Panel 1 | | Apheresis Panel 2 | |
|---|---|---|---|
| Antibody | Rationale | Antibody | Rationale |
| CD3 | Pan T-cell marker | CD3 | Pan T-cell marker |
| CD4 | Helper T-cell marker | CXCR3 | Chemokine-effector |
| | | | recruitment |
| CD8 | Cytotoxic T-cell | CD8 | Cytotoxic T-cell |
| | marker | | marker |
| CD66b | Granulocyte marker | CCR7 | Differentiation T-cell |
| | | | marker |
| CD19 | B-cell marker | CD45RA | Differentiation T-cell |
| | | | marker |
| CD14 | Monocyte/macrophage | CD27 | Activation marker |
| | marker | | |
| CD56 | NK cell marker | CD28 | Activation marker |
| | | CD95 | Activation marker |
| | | CD122 | Differentiation marker |
| | | | (IL-2 receptor) |

Flow cytometry assays are performed for evaluation of transduction efficiency, and also to evaluate phenotype and T-cell activation status of KTE-C19 product samples that have been released for patient infusion. Cyropreserved pre-infusion product is assessed with product Panels 1-3 summarized in Table 3.

TABLE 3
Flow cytometry panel for characterization of pre-infusion product

| Apheresis Panel 1 | | Apheresis Panel 2 | | Apheresis Panel 3 | |
|---|---|---|---|---|---|
| Antibody | Rationale | Antibody | Rationale | Antibody | Rationale |
| CD3 | Pan T-cell marker | CD3 | Pan T-cell marker | CD3 | Pan T-cell marker |
| CD8 | Cytotoxic T-cell | CD8 | Cytotoxic T-cell | CD8 | Cytotoxic T-cell |
| | marker | | marker | | marker |
| CD45RA | Differentiation T-cell | CD45RA | Differentiation | CD45RA | Differentiation |
| | marker | | T-cell marker | | T-cell marker |
| CCR7 | Differentiation T-cell | CCR7 | Differentiation | CCR7 | Differentiation |
| | marker | | T-cell marker | | T-cell marker |
| CD122 | Differentiation | CD57 | Activation marker | CD25 | Activation marker |
| | marker(IL-2 receptor) | | | | |
| CD27 | Activation marker | CD107α | Activation marker | CD69 | Activation marker |
| CD28 | Activation marker | CD279 | Activation marker | CD137 | Activation marker |
| | | | (PD-1) | | (4-1BB) |
| CD95 | Activation marker | CD19 CAR | KTE-C19 | CD19 CAR | KTE-C19 |
| | | | identification | | identification |
| CD19 | KTE-C19 | | | | |
| CAR | identification | | | | |

Flow cytometry assays are used for evaluation of surface expression of several key markers as they relate to phenotype and activation of longitudinal patient PBMCs using PBL Panels 1-4 shown in Table 4. This data are used to monitor KTE-C19 expansion, persistence and phenotype post infusion. In addition to CAR level monitoring, panel 4 (Table 4) is designed to interrogate levels on PBMC populations that are impacted by conditioning and on-target off-tumor CAR activity (i.e., normal B-cells).

Longitudinal patient blood samples are processed into cryopreserved PBMC. Cryopreserved longitudinal patient PBMC is collected at Day-5, Day0, Day7, Wk2, Wk4 (On Wk4 prior to utomilumab then Day30 and Day 36). To align with utomilumab administration, additional blood collection includes draws every 4 weeks prior to utomilumab, 2 and 6 days after each utomilumab administration. In the long-term follow-up blood is drawn every 3 months up to 2 years to monitor immune reconstitution.

TABLE 4
Flow cytometry panel for evaluation of post-infusion PBMC

| Apheresis Panel 1 | | Aphresis Panel 2 | |
|---|---|---|---|
| Antibody | Rationale | Antibody | Rationale |
| CD3 | Pan T-cell marker | CD3 | Pan T-cell marker |
| CD8 | Cytotoxic T-cell marker | CD8 | Cytotoxic T-cell marker |
| CD45RA | Differentiation T-cell marker | CD45RA | Differentiation T-cell marker |
| CCR7 | Differentiation T-cell marker | CCR7 | Differentiation T-cell marker |
| CD122 | Differentiation marker(IL-2 receptor) | CD57 | Activation marker |
| CD27 | Activation marker | CD107α | Activation marker |
| CD28 | Activation marker | CD279 | Activation marker (PD-1) |
| CD95 | Activation marker | CD19 CAR | KTE-C19 identification |
| CD19 CAR | KTE-C19 identification | | |
| Apheresis Panel 3 | | Apheresis Panel 4 | |
| Antibody | Rationale | Antibody | Rationale |
| CD3 | Pan T-cell marker | CD3 | Pan T-cell marker |
| CD8 | Cytotoxic T-cell marker | CD4 | Helper T-cell marker |
| CD45RA | Differentiation T-cell marker | CD8 | Cytotoxic T-cell marker |
| CCR7 | Differentiation T-cell marker | CD66b | Granulocyte marker |
| CD25 | Activation marker | CD19 | B-cell marker |
| CD69 | Activation marker | CD14 | Monocyte/macrophage marker |
| CD137 | Activation marker (4-1BB) | CD56 | NK cell marker |
| CD19 CAR | KTE-C19 identification | CD19 CAR | KTE-C19 identification |
| | | BD | For determining absolute counts |
| | | Trucount | of leucocytes in the blood |

A quantitative polymerase chain reaction (qPCR) assay can be used for longitudinal monitoring of anti-CD19 CAR T-cell presence, expansion and persistence in peripheral blood. Post-infusion cryopreserved PBMC is utilized to monitor levels and clearance of gene marked cells over time. Cryopreserved longitudinal patient PBMC is collected at Day-5, Day0, Day7, Wk2, Wk4 (On Wk 4 prior to utomilumab then Day30 and Day 36). To align with utomilumab administration, additional blood collection includes draws every 4 weeks prior to utomilumab, 2 and 6 days after each utomilumab administration. In the long-term follow-up blood are drawn every 3 months for up to 2 years to monitor for the presence of persisting anti-CD19 CAR T-cells.

A co-culture assay for detailed anti-CD19 CAR product characterization is used. A targeted 44 analyte MSD®, Luminex® and Quantikine® ELISA approach along with multi-parameter flow cytometry is utilized for evaluation of cytokine production and T-cell activation status (Table 5). Sample types include cryopreserved product, K562 cells engineered to express CD19 (CAR target), and K562 cells engineered to express NGFR (evidence of off-target activity). T-cells harvested from co-culture are analyzed using the product characterization panel described in Table 3.

TABLE 5
Cytokine co-culture panel (MSD ®, Luminex ®)

| Immune | Inflammatory | Immune | | |
|---|---|---|---|---|
| homeostatic | cytokines and | modulating | | Immune |
| cytokines | markers | cytokines | Chemokines | effectors |
| IL-15 | IL-6 | IL-13 | IL-8 | Granzyme A |
| IL-7 | IL-1α | IL-4 | MCP-1 | Granzyme B |
| IL-2 | IL-1β | IL-5 | MCP-4 | sFASL |
| | IL-17α | IL-10 | MIP-1α | Perforin |
| | TNFα | IFN-y | MIP-1β | sCD137 |
| | TNFβ | IL-12p40 | IP-10 | |
| | GM-CSF | IL-12p70 | TARC | |
| | | IL-16 | Eotaxin | |
| | | | Eotaxin-3 | |
| | | | MDC | |

Multi-parametric assays can be used for evaluation of longitudinal serum chemokine, cytokine and immune effector levels to monitor serum analyte expression changes in the context of anti-CD19 CAR T-cell expansion, phenotype and persistence. Objective response features and safety correlates are be evaluated in relation to observed changes in serum analytes. Longitudinal patient serum samples are processed and cryopreserved. Longitudinal serum samples (Day-5, Day0, Q3D beginning on day 1 and then every other day through hospitalization, Wk2, Wk4) and additional blood collection includes draws every 4 weeks prior to utomilumab, 2 and 6 days after each utomilumab administration to align with utomilumab administration. Evaluation can include the analytes described in Table 6.

TABLE 6
Serum analyte panel (MSD ®, Luminex ® and Quantikine ®)

| Immune | Inflammatory | Immune | | | | |
|---|---|---|---|---|---|---|
| homeostatic | cytokines | modulating | | Immune | Angiogenic | |
| cytokines | and markers | cytokines | Chemokines | effectors | cytokines | Other |
| IL-15 | IL-6 | IL-13 | IL-8 | Granzyme A | FGF-2 | IL1Rα |
| IL-7 | IL-1α | IL-4 | MCP-1 | Granzyme B | sICAM-1 | IL1Rβ |
| IL-2 | IL-1β | IL-5 | MCP-4 | sFASL | sVCAM-1 | Ferritin |
| | IL-17α | IL-10 | MIP-1α | Perforin | VEGF | |
| | TNFα | IFN-γ | MIP-1β | | VEGF-C | |
| | TNFβ | IL-12p40 | IP-10 | | VEGF-D | |
| | GM-CSF | IL-12p70 | TARC | | PLGF | |
| | CRP | IL-16 | Eotaxin | | | |
| | SAA | | Eotaxin-3 | | | |
| | | | MDC | | | |

Patient serum samples are evaluated pre-infusion (baseline), at Day 28 and 3 Months post-infusion for anti-KTE-C19 or anti-utomilumab antibodies. Serum samples that show evidence of anti-KTE-C19 and/or anti-utomilumab antibody formation are evaluated for presence of neutralizing antibody formation.

Cerebrospinal fluid (CSF) as well as any additional subject samples (e.g., pleural fluid) can be collected from patients who develop neurologic toxicity or CRS to enable evaluation of levels of inflammatory cytokines and chemokines and levels and phenotypes of infiltrating anti-CD19 CAR T-cells. Flow cytometry and MSD/Luminex panels previously described are leveraged for this evaluation.

A summary of the sample analysis to be performed is provided in Table 7.

TABLE 7
Sample analysis plan

| Item | Sample Analyses to be Performed |
|---|---|
| 1 | BLOOD: T and B-cells; Flow; Longitudinal PBMC |
| 2 | BLOOD: CAR-T-cells Flow; Longitudinal PBMC |
| 3 | BLOOD: CYTOKINES (MSD and Luminex); |
| | Longitudinal serum/plasma |
| 4 | BLOOD: CAR-T-cells |
| | PCR; Longitudinal PBMC |
| 5 | Serum/plasma: sCD137; Baseline and longitudinal |
| 6 | FFPE: IHC (target and exploratory) CD3, Ki-67, |
| | CD8, CD137, PD1 and PDL1 expression on |
| | tumor and tumor infiltrating immune cells |
| | including CAR-T-cells and stroma; |
| | Baseline and Post-dose biopsy |
| 7 | FFPE: Molecular classification: COO |
| | (prefer Gene signature over Hans); Baseline |
| 8 | FFPE: Markers of CAR-T-cell activity; Baseline and |
| | Post-dose biopsy |
| 9 | FFPE: RNA (transcript analysis); Baseline and |
| | Post-dose biopsy |
| 10 | FFPE: Immunological score (IHC); Baseline and |
| | Post-dose biopsy |
| 11 | FFPE: Molecular classification: subtypes: BCL2/MYC |
| | D/T positive; Baseline (FISH and IHC) |
| 12 | FFPE: DNA (sequencing); Baseline and Post-dose biopsy |
| 13 | TCR/BCR sequencing |

### Example 3: Paired Tissue Biopsy Analysis

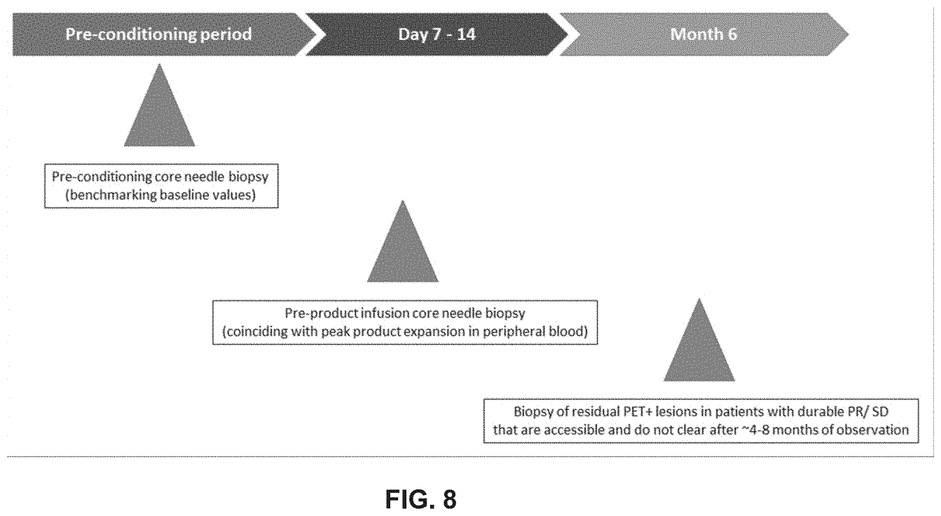

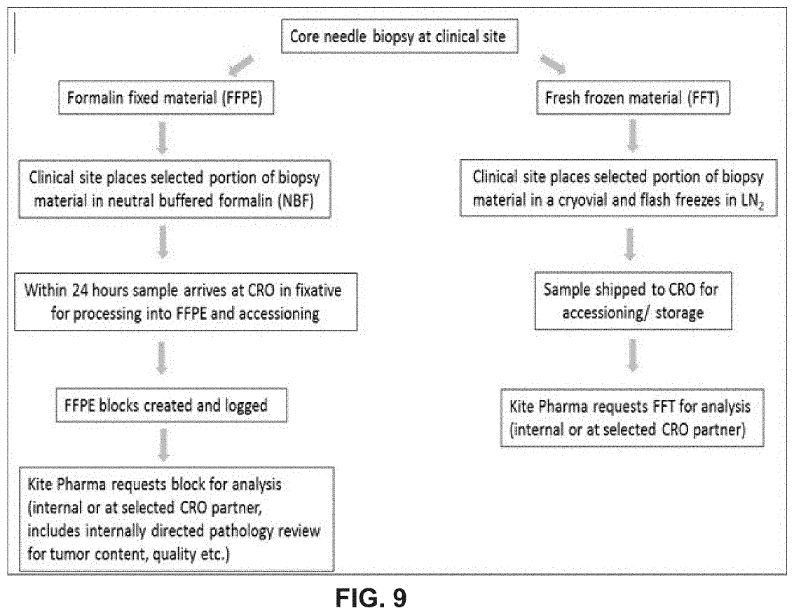

Collection of core needle tumor biopsies occurs at baseline (pre-conditioning) and post-product T-cell infusion, at or around day 7-14 and largely coinciding with the product expansion peak in blood. The paired biopsy collection schedule is shown in FIG. 8. Core needle biopsy FFPE will be created in 120 mL jars containing 60 mL of neutral buffered formalin (fixative for FFPE), 1.5 mL cryovials (FFT) and appropriate labels. Core needle biopsy material is placed into fixative (3-4 cores) for processing into FFPE. Remaining cores (1-2) are placed immediately into a 1.5 mL cryovial for flash freezing in liquid nitrogen (LN₂) or dry ice/ethanol slurry. Samples can be stored at −80° C. FIG. 9 summarizes sample processing schemes for core needle biopsies.

The following analysis is performed on paired tissue biopsy to assess the products antitumor effect in the context of refractory large B-cell lymphoma or r/r DLBCL:

### IHC—Immune Infiltrate

CD19 CAR detection (in situ hybridization approach, FISH and ISH)
CD25 and CD107α (evidence of CAR activation and degranulation)
Ki-67 (evidence of intra-tumoral CAR expansion)
PD-1 (evidence of CAR exhaustion)

### IHC—Tumor

CD19 (CAR target antigen)
CD22 (prevalence in CD19 negative lesions)
PD-L1/2 (checkpoint mediated resistance)
Caspase 3 (evidence of CAR directed tumor cell killing)

### Additional IHC Analysis/Goals

Assessment of CAR T-cell/tumor cell proximity
CAR product detection development
Development of correlative imaging resource partner

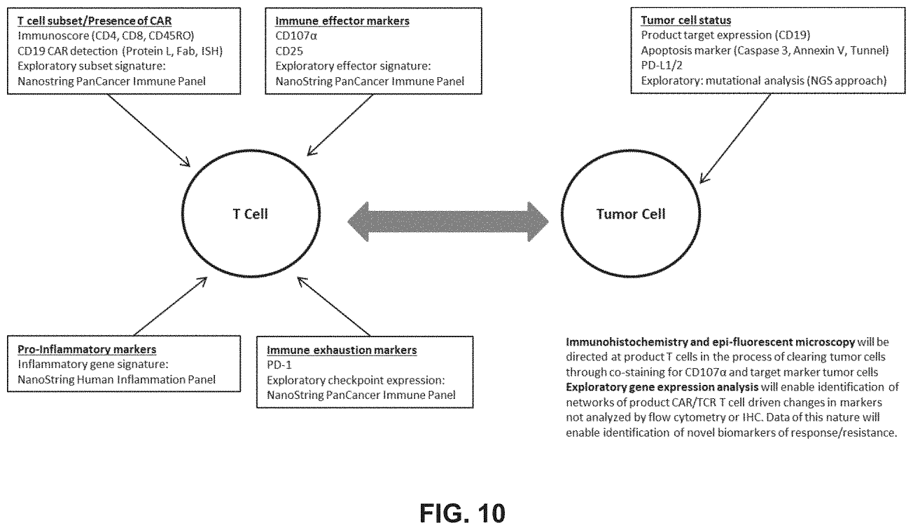

Additionally, transcript analyses and tumor sequencing can be performed using NanoString (Immunosign™) for gene expression analysis using either fixed (i.e., FFPE) or fresh sample formats. Commercially available code sets have been developed to determine expression patterns in immune infiltrate (PanCancer Immune panel—infiltrate composition, evidence of checkpoint regulation) and also markers of inflammation (Human Inflammation panel—additional markers to provide evidence of activation). Creation of custom “fit for purpose” panels can be designed. Alternatively, higher content microarray can be pursued to expand the scope of genes analyzed for expression (i.e., Almac or Agilent high content microarray platforms). FIG. 10 shows a schematic view of markers and analysis approaches that can be employed to evaluate patient biopsy samples.

IHC analysis is utilized to determine the presence, phenotype and function of product T-cells, tumor tissue expression of product target, and their relative micro-environmental localization. Prolonged presence of activated T-cells within the tumor tissue would indicate a long term, localized immune reaction or immune mediated confinement of tumor, as primary mechanism of action for durable PRs. Presence of CAR negative T-cells within such lesions can suggest a potential employment of endogenous T-cells recognizing unrelated tumor targets.

Presence of an anti-inflammatory tumor microenvironment (Treg presence, upregulated PD-L1/2 tumor expression, etc.) may be assessed by analyzing biopsies from relapsing or new lesions. A target expression analysis, along with other markers (i.e., CD22 or other relevant CD antigens) as well as the full Hans algorithm including monitoring of dysregulation of c-myc, bcl-2, bcl-6 (relevant to NHL indication), to document tumor evolution, potential target loss and expression of other targets can be performed. Additionally, analysis of product T-cell presence and phenotype within tumor lesions may be determined.

Single-cell transcript analysis can be performed using pre-infusion product (antigen naïve and experienced) and cryopreserved longitudinal patient PBLs (e.g. from Day-5, Day0, Day7, Wk2, Wk4, Mth3, Mth6, Mth9, Mth12, Mth15, Mth18, Mth24, Mth36, Mth48, Mth60, Mth72 and annually thereafter as applicable). Pre-infusion product and longitudinal PBLs are analyzed at the single cell level for expression patterns of RNA transcripts using a rationally designed assay panel. Panel design includes markers for CAR T-cell identification and also markers of lineage, activation, and exhaustion.

### Example 4: Minimal Residual Disease (MRD) and BCR/TCR Monitoring

### Minimal Residual Disease (MRD)

Adaptive Biotechnologies ClonoSIGHT® technology is used to measure MRD in a highly sensitive manner. Evaluation of circulating tumor DNA (ctDNA) at diagnosis and during the course of therapy using Adaptive's high-throughput sequencing platform for identification and measurement of tumor specific immunoglobulin genes is evaluated in a pre-treatment sample followed by longitudinal monitoring. Evaluation of genetic markers of disease has a sensitivity of 10e-6 and is performed utilizing patient peripheral blood. This approach may demonstrate superior monitoring of disease relative to CT imaging and also molecular disease clearance when a CR is determined.

Adaptive (peripheral blood) samples collected at enrollment period (calibration), aspirate initiated at time of OR evaluation, longitudinally every 3 months up to 1 year, month 18 and month 24 are used to support MRD in subjects determined to have undergone a complete response (CR).

### BCR/TCR Monitoring

Adaptive Biotechnologies ImmunoSEQ® technology is used to characterize B-cell receptor (BCR) diversity in serial refractory large B-cell lymphoma or DLBCL biopsies (pre-infusion, post-infusion and upon relapse). Evaluation of BCR diversity may be used to identify or confirm malignant clones during the course of therapy, as well as confirm relapse of the original tumor as opposed to secondary malignancies. Secondly, BCR sequencing of peripheral blood lymphocytes is used to confirm recovery of normal B-cell repertoire.

TCR diversity evaluation in pre-infusion product, post-infusion blood and serial biopsies is used to understand T-cell diversity changes during the course of treatment. Monitoring expansion of CAR T specific TCR sequences originally present in product that expand and become dominant in blood or tumor lesions may inform on the presence and nature of reactive T-cell clones that play a significant role in tumor clearance. Data of this nature may be used to identify T-cell clones that preferentially expand and eradicate tumor cells by “epitope spreading” mechanisms, involving reactivity against unrelated epitopes such as the ones associated with neoantigens.

Sample types and timing required to support MRD in subjects determined to have undergone a complete response (CR) for BCR Sequencing include pre-infusion tumor biopsy, post-infusion tumor biopsy (Day 7-14), relapse tumor biopsy, longitudinal PBMC (as applicable Mth3, Mth6, Mth12, Mth18, Mth24).

Sample types and timing required to support MRD in subjects determined to have undergone a complete response (CR) for TCR Sequencing include pre-infusion tumor biopsy, post-infusion tumor biopsy (Day 7-14), relapse tumor biopsy, product CAR T-cells, apheresed T-cells, longitudinal PBMC (Dy14, Dy28, Mth3, Mth6 and Mth12).

### Example 5

This study examined the effect of Utomilumab to the anti-CD19 CAR T-cells. In this study, the cells were incubated with a tool antibody, which was previously shown to stimulate or activate the CAR T-cells; in the presence or absence of Utomilumab. The production or levels of several cytokines, chemokines, and effector molecules (analytes) were used to evaluate the potential effects. Anti-CD19 CAR T-cells were generated from peripheral blood mononuclear cells of healthy subjects (A, B, C, D, and E). The anti-CD19 CAR T-cells in R10 media (1×10⁶ cells/mL) were incubated overnight at 37° C. and 5% CO₂. The 96-well plates were coated with the tool antibody (0.33 μg/mL), Utomilumab (titration concentration from 0 to 100 μg/mL by a 3-fold dilution) or a control antibody which does not bind to 4-1BB (titration concentration from 0 to 100 μg/mL by a 3-fold dilution) overnight at 4° C. The coated plates were washed twice using R10 media (RPMI 1640 with 10% FBS) and added with 1×10⁵ anti-CD19 CAR T-cells. The total final volume of each well was adjusted to 2004 using R10 media. Following overnight incubation at 37° C. and 5% CO₂, the supernatants were harvested and analyzed using the MILLIPLEX MAP Human CD8⁺ T Cell Magnetic Bead Panel Premixed 17 Plex—Immunology Multiplex Assay. The peak fold change was calculated by dividing the analyte output in the presence of Utomilumab by those in the presence of the control antibody at the corresponding concentration. The peak fold difference across the titration concentration for each analyte is shown in Table 8.

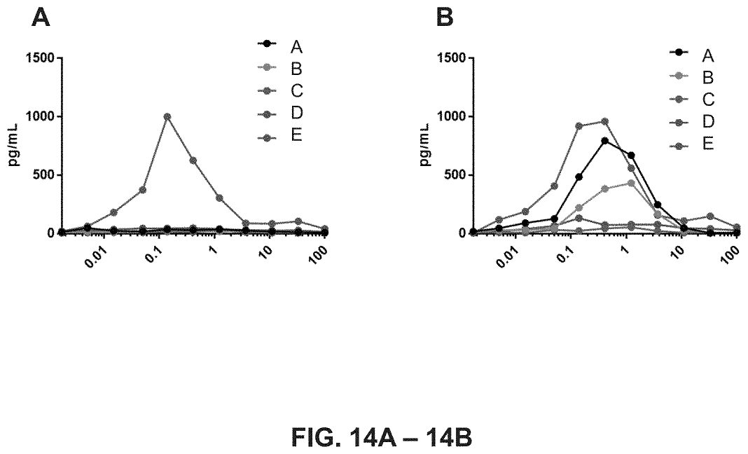

Results showed that, in the presence of Utomilumab, the levels or production of several cytokines, chemokines and effector molecules by the anti-CD19 CAR T-cells (that were stimulated by the tool antibody) were increased (Table 8). The levels or production of IL-2 were increased 1.9-25.9 fold compared to those in the presence of the control antibody, except subject E where a non-specific increase of IL-2 production was observed (FIG. 14). The IL-2 production was below the limit of quantification in the cells that were incubated with Utomilumab only or the control antibody only, in each case without the tool antibody (triplicate data not shown). This shows that Utomilumab alone did not stimulate anti-CD19 CAR T-cells to produce IL-2.

All publications, patents, patent applications, sequences under cited database accession numbers, and references, including prescribing information, that are mentioned in this specification are herein incorporated by reference to the same extent as if each individual publication, patent, or patent application was specifically and individually indicated to be incorporated by reference. However, the citation of a reference herein should not be construed as an acknowledgement that such reference is prior art to the present invention. To the extent that any of the definitions or terms provided in the references incorporated by reference differ from the terms and discussion provided herein, the present terms and definitions control.

TABLE 8
Peak fold change of the analyte production by anti-CD19 CAR-T cells

| Subject | GM-CSF | sCD137 | IFN-γ | IL-10 | Granzyme A | IL-13 | Granzyme B | sFAS | IL-2 |
|---|---|---|---|---|---|---|---|---|---|
| A | 1.7 | 2.4 | 1.1 | 6.1 | 1.0 | 2.4 | 1.0 | 1.0 | 25.9 |
| B | 10.4 | 4.4 | 6.9 | 20.4 | 2.6 | 14.3 | 15.4 | 1.0 | 23.7 |
| C | 3.3 | 2.2 | 3.9 | 3.0 | 1.7 | 2.0 | 1.0 | 1.0 | 3.2 |
| D | 2.0 | 1.4 | 2.0 | 3.5 | 1.2 | 1.7 | 2.2 | 1.0 | 2.9 |
| E | 1.6 | 1.8 | 1.9 | 1.9 | 1.4 | 1.7 | 1.9 | 1.0 | 1.9 |

| Subject | IL-4 | IL-5 | lL-6 | sFASL | MIP-1α | MIP-1β | TNF-α | Perforin |
|---|---|---|---|---|---|---|---|---|
| A | 5.2 | 4.8 | 2.3 | 2.5 | 1.0 | 1.0 | 1.0 | 1.5 |
| B | 10.3 | 24.6 | 2.1 | 3.7 | 5.5 | 5.0 | 5.8 | 2.1 |
| C | 5.7 | 132.3 | 1.9 | 2.0 | 1.0 | 2.5 | 1.9 | 1.5 |
| D | 2.5 | 2.2 | 1.0 | 1.7 | 2.1 | 1.7 | 1.7 | 1.5 |
| E | 1.8 | 1.7 | 1.3 | 1.5 | 1.7 | 1.7 | 1.9 | 1.1 |

### Sequences and SEQ ID Numbers

The instant disclosure comprises a number of polypeptide sequences. For convenience, Table 9 below correlates each sequence with its corresponding description and SEQ ID NO.

TABLE 9
SEQ ID Numbers

| | | |
|---|---|---|
| Utomilumab VH | SEQ ID NO: 1 | EVQLVQSGAEVKKPGESLRISCKGSGYSFSTYWI |
| | | SWVRQMPGKGLEWMGKIYPGDSYTNYSPSFQG |
| | | QVTISADKSISTAYLQWSSLKASDTAMYYCARG |
| | | YGIFDYWGQGTLVTVSS |
| Utomilumab HC | SEQ ID NO: 2 | EVQLVQSGAEVKKPGESLRISCKGSGYSFSTYWI |
| | | SWVRQMPGKGLEWMGKIYPGDSYTNYSPSFQG |
| | | QVTISADKSISTAYLQWSSLKASDTAMYYCARG |
| | | YGIFDYWGQGTLVTVSSASTKGPSVFPLAPCSRS |
| | | TSESTAALGCLVKDYFPEPVTVSWNSGALTSGV |
| | | HTFPAVLQSSGLYSLSSVVTVPSSNFGTQTYTCN |
| | | VDHKPSNTKVDKTVERKCCVECPPCPAPPVAGP |
| | | SVFLFPPKPKDTLMISRTPEVTCVVVDVSHEDPE |
| | | VQFNWYVDGVEVHNAKTKPREEQFNSTFRVVS |
| | | VLTVVHQDWLNGKEYKCKVSNKGLPAPIEKTIS |
| | | KTKGQPREPQVYTLPPSREEMTKNQVSLTCLVK |
| | | GFYPSDIAVEWESNGQPENNYKTTPPMLDSDGSF |
| | | FLYSKLTVDKSRWQQGNVFSCSVMHEALHNHY |
| | | TQKSLSLSPGK |
| Utomilumab VL | SEQ ID NO: 3 | SYELTQPPSVSVSPGQTASITCSGDNIGDQYAHW |
| | | YQQKPGQSPVLVIYQDKNRPSGIPERFSGSNSGN |
| | | TATLTISGTQAMDEADYYCATYTGFGSLAVFGG |
| | | GTKLTVL |
| Utomilumab LC | SEQ ID NO: 4 | SYELTQPPSVSVSPGQTASITCSGDNIGDQYAHW |
| | | YQQKPGQSPVLVIYQDKNRPSGIPERFSGSNSGN |
| | | TATLTISGTQAMDEADYYCATYTGFGSLAVFGG |
| | | GTKLTVLGQPKAAPSVTLFPPSSEELQANKATLV |
| | | CLISDFYPGAVTVAWKADSSPVKAGVETTTPSKQ |
| | | SNNKYAASSYLSLTPEQWKSHRSYSCQVTHEGS |
| | | TVEKTVAPTECS |
| Utomilumab | SEQ ID NO: 5 | STYWIS |
| H-CDR1 | | |
| Utomilumab | SEQ ID NO: 6 | KIYPGDSYTNYSPSFQG |
| H-CDR2 | | |
| Utomilumab | SEQ ID NO: 7 | RGYGIFDY |
| H-CDR3 | | |
| Utomilumab | SEQ ID NO: 8 | SGDNIGDQYAH |
| L-CDR1 | | |
| Utomilumab | SEQ ID NO: 9 | QDKNRPS |
| L-CDR2 | | |
| Utomilumab | SEQ ID NO: 10 | ATYTGFGSLAV |
| L-CDR3 | | |

1. A method of treating a B-cell lymphoma or leukemia in a patient in need thereof comprising administering a CD19-directed genetically modified T-cell immunotherapy and a 4-1BB (CD137) agonist.

2. The method of claim 1, wherein the CD19-directed genetically modified T-cell immunotherapy is an autologous or allogenic immunotherapy.

3. The method of claim 1, wherein the T-cells are genetically modified ex vivo.

4. The method of claim 1, wherein the T-cells are genetically modified by viral transduction, retroviral transduction or lentiviral transduction.

5. The method of claim 1, wherein the CD19-directed genetically modified T-cell immunotherapy, is genetically modified to express a chimeric antigen receptor (CAR) said CAR comprising an anti-CD19 single chain variable fragment (scFv) linked to CD28 and CD3-zeta co-stimulatory domains.

6. The method of claim 1, wherein the CD19-directed genetically modified T-cell immunotherapy is axicabtagene ciloleucel.

7. The method of claim 1, wherein the 4-1BB (CD137) agonist is an antigen binding molecule or fragment thereof.

8. The method of claim 1, wherein the 4-1BB (CD137) agonist is an isolated antibody, or antigen-binding portion thereof, comprising three CDRs of a VH region amino acid sequence as set forth in SEQ ID NO:1 and three CDRS of a VL region amino acid sequence set forth in SEQ ID NO: 3

9. The method of claim 1, wherein the 4-1BB (CD137) agonist is an isolated antibody, or antigen-binding portion thereof, comprising: (a) a H-CDR1 as set forth in SEQ ID NO:5; (b) a H-CDR2 as set forth in SEQ ID NO:6; (c) a H-CDR3 as set forth in SEQ ID NO:7; (d) a L-CDR1 as set forth in SEQ ID NO:8; (e) a L-CDR2 as set forth in SEQ ID NO:9; and (f) a L-CDR3 as set forth in SEQ ID NO:10.

10. The method of claim 1, wherein the 4-1BB (CD137) agonist is a fully human monoclonal antibody.

11. The method of claim 1, wherein the 4-1BB (CD137) agonist comprises a VH region amino acid sequence as set forth in SEQ ID NO:1 and a VL region amino acid sequence set forth in SEQ ID NO: 3.

12. The method of claim 1, wherein the 4-1BB (CD137) agonist comprises a heavy chain amino acid sequence as set forth in SEQ ID NO:2 and a light chain amino acid sequence as set forth in SEQ ID NO:4, with the proviso that the C-terminal lysine residue of SEQ ID NO:2 is optionally absent.

13. The method of claim 1, wherein the 4-1BB (CD137) agonist is utomilumab.

14. The method of claim 1, wherein the B-cell lymphoma or leukemia is selected from the group consisting of Acute Lymphoblastic Leukemia (ALL), AIDS-related lymphoma, ALK-positive large B-cell lymphoma, Burkitt's lymphoma, Chronic lymphocytic leukemia, CLL), Classical Hodgkin lymphoma, Diffuse large B-cell lymphoma (DLBCL), Primary Mediastinal Large B-cell Lymphoma (PMBCL), Follicular lymphoma, Intravascular large B-cell lymphoma, Large B-cell lymphoma arising in HHV8-associated multicentric Castleman's disease, Lymphomatoid granulomatosis, Lymphoplasmacytic lymphoma, Mantle cell lymphoma (MCL), Marginal zone B-cell lymphoma (MZL), Mucosa-Associated Lymphatic Tissue lymphoma (MALT), Nodal marginal zone B-cell lymphoma (NMZL), Nodular lymphocyte predominant Hodgkin's lymphoma, Non-Hodgkin's lymphoma, Plasmablastic lymphoma, Primary central nervous system lymphoma, Primary effusion lymphoma, Splenic marginal zone lymphoma (SMZL), and Waldenström's macroglobulinemia, relapsed or refractory large B-cell lymphoma, diffuse large B-cell lymphoma (DLBCL) not otherwise specified, high grade B-cell lymphoma, and DLBCL arising from follicular lymphoma.

15.-17. (canceled)

18. The method of claim 1, wherein the CD19-directed genetically modified T-cell immunotherapy is administered to the patient by intravenous infusion at a dose between about 1×10⁶ and about 2×10⁶ CAR-positive viable T-cells per kg body weight up to a maximum dose of about 1×10⁸ CAR-positive viable T-cells.

19.-21. (canceled)

22. The method of claim 1, wherein the 4-1BB (CD137) agonist is administered at a dose ranging from about 1 mg to about 200 mg.

23. The method of claim 22, wherein the 4-1BB (CD137) agonist is administered at a dose of about 1 mg, about 10 mg, about 30 mg, about 100 mg or about 200 mg.

24. The method of claim 1 wherein the CD19-directed genetically modified T-cell immunotherapy and the 4-1BB (CD137) agonist are administered simultaneously.

25. The method of claim 1, wherein the CD19-directed genetically modified T-cell immunotherapy is administered prior to the 4-1BB (CD137) agonist.

26. (canceled)

27. The method of claim 1, wherein the CD19-directed genetically modified T-cell immunotherapy is administered after the 4-1BB (CD137) agonist.

28. (canceled)

29. The method of claim 1, wherein the 4-1BB (CD137) agonist is administered about every 4 weeks.

30. The method claim 1, wherein the patient is administered a conditioning chemotherapy regimen prior to administration of the CD19-directed genetically modified T-cell immunotherapy and the 4-1BB (CD137) agonist.

31. The method of claim 1 further comprising monitoring the patient following administration for signs and symptoms of an adverse reaction.

32. The method of claim 31, wherein the adverse reaction is selected from the group consisting of cytokine release syndrome (CRS), a neurologic toxicity, a hypersensitivity reaction, a serious infection, a cytopenia and hypogammaglobulinemia.

33. The method of claim 1 further comprising monitoring the patient following administration for changes in markers of phenotype and activation of patient peripheral blood mononuclear cells (PBMCs).

34. The method of claim 33, wherein the markers of phenotype and activation of patient PBMCs comprise a pan T-cell marker, cytotoxic T-cell marker, differentiation T-cell marker, differentiation marker, IL-2 receptor, activation marker, PD1, 4-1BB, helper T-cell marker, granulocyte marker, B-cell marker, monocyte/macrophage marker, NK cell marker, and/or axicabtagene ciloleucel identification.

35. The method of claim 34, wherein the markers of phenotype and activation of patient PBMCs are monitored by a panel comprising antibodies to CD3, CD4, CD8, CD45RA, CCR7, CD122, CD27, CD28, CD95, CD57, CD107a, CD279, CD25, CD69, CD137, CD66b, CD19, CD14, CD56 and/or CD19 CAR.

36. (canceled)

37. The method of claim 1 further comprising monitoring the patient serum following administration for chemokine, cytokine and/or immune effector levels.

38. The method of claim 37, wherein the patient serum is monitored for IL-15, IL-7, IL-2, IL-6, IL1α, IL-1β, IL-17α, TNFα, TNFβ, GM-CSF, CRP, SAA, IL-13, IL-4, IL-5, IL-10, IFNγ, IL-12p40, IL-12p70, IL-16, IL-8, MCP-1, MCP-4, MIP-1α, MIP-1β, IP-10, TARC, Eotaxin, Eotaxin-3, MDC, Granzyme A, Granzyme B, sFASL, Perforin, FGF-2, sICAM-1.

39.-43. (canceled)

44. A method of treating a B-cell lymphoma or leukemia in a patient in need thereof comprising:
(a) administering to the patient a CD19-directed genetically modified T-cell immunotherapy;
(b) administering to the patient a 4-1BB (CD137) agonist; and
(c) monitoring the patient following administration for signs and symptoms of an adverse reaction.

45. (canceled)

46. A method of treating refractory diffuse large B-cell lymphoma in a patient in need thereof comprising:
(a) administering to the patient a CD19-directed genetically modified T-cell immunotherapy;
(b) administering to the patient a 4-1BB (CD137) agonist; and
(c) monitoring the patient serum following administration for chemokine, cytokine and/or immune effector levels.

47. (canceled)


SEQUENCE LISTING (10 sequences)

SEQ ID NO: 1 - protein, 116 aa, Homo sapiens
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 2 - protein, 442 aa, Homo sapiens
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 3 - protein, 108 aa, Homo sapiens
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 4 - protein, 214 aa, Homo sapiens
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 6 aa, Homo sapiens
STYWIS

SEQ ID NO: 6 - protein, 17 aa, Homo sapiens
KIYPGDSYTNYSPSFQG

SEQ ID NO: 7 - protein, 8 aa, Homo sapiens
RGYGIFDY

SEQ ID NO: 8 - protein, 11 aa, Homo sapiens
SGDNIGDQYAH

SEQ ID NO: 9 - protein, 7 aa, Homo sapiens
QDKNRPS

SEQ ID NO: 10 - protein, 11 aa, Homo sapiens
ATYTGFGSLAV